(19) United States
(12) Patent Application Publication
(10) Pub. No.: US 2017/0233409 A1
(43) Pub. Date: Aug. 17, 2017

(54) TYPE IV SECRETION SYSTEM INHIBITORS

(71) Applicants: Maria Hadjifrangiskou, Brentwood, TN (US); Fredrik Almqvist, Umeå (SE); Carrie L. Shaffer, Nashville, TN (US); James Good, Umeå (SE)

(72) Inventors: Maria Hadjifrangiskou, Brentwood, TN (US); Fredrik Almqvist, Umeå (SE); Carrie L. Shaffer, Nashville, TN (US); James Good, Umeå (SE)

(21) Appl. No.: 15/436,094

(22) Filed: Feb. 17, 2017

Related U.S. Application Data
(60) Provisional application No. 62/296,553, filed on Feb. 17, 2016.

(51) Int. Cl. C07D 513/04; C07H 15/26

(52) U.S. Cl. CPC C07D 513/04; C07H 15/26

(57) ABSTRACT

Described are compounds, compositions, and methods for treating or preventing conditions or disorders caused by or associated with bacterial type IV secretion systems.

### RELATED APPLICATIONS

This application claims the benefit of U.S. provisional application Ser. No. 62/296,553, filed Feb. 17, 2016, the entire contents of which are hereby incorporated by reference.

### TECHNICAL FIELD

The present disclosure relates to compounds, compositions, and methods for treating or preventing conditions or disorders caused by or associated with bacteria, including bacterial type IV secretion systems.

### BACKGROUND

Numerous bacterial species translocate effector molecules into target cells to subvert host defense mechanisms and hijack cellular processes. Delivery of bacterial effectors can be achieved using elaborate secretion systems that are assembled in response to specific environmental stimuli, such as direct bacterial contact with target cells. Type IV secretion systems (T4SS) are extraordinarily versatile contact-dependent cargo delivery systems that are both phylogenetically and functionally diverse. These membrane-spanning systems are composed of conserved core complex subunits, as well as species-specific components that afford apparatus specialization and facilitate occupation of specific intracellular and extracellular niches.

T4SSs can be divided into three subfamilies: (i) DNA conjugation machines, (ii) DNA uptake/release systems that exchange DNA with the extracellular milieu, and (iii) effector translocator systems. T4SSs contribute to the pathogenesis of disease caused by several human pathogens, including Brucella, Bartonella, Coxiella, Rickettsia, Legionella pneumophila, Helicobacter pylori, and phytopathogens such as Agrobacterium tumefaciens. Studies of the prototypical vir T4SS effector translocator system in Agrobacterium tumefaciens have laid the groundwork for the study of T4SSs in other bacterial species including the distantly related cag T4SS that is harbored by virulent strains of the gastric bacterium H. pylori.

H. pylori can persist within the human gastric mucosa, often for the lifetime of the individual. Although the majority of H. pylori-infected individuals remain asymptomatic, colonization by H. pylori is associated with a broad range of clinical outcomes ranging from non-atrophic gastritis to severe disorders including gastric and duodenal ulcers and gastric adenocarcinoma. These severe gastric diseases occur more frequently in individuals who are colonized by H. pylori strains that produce a T4SS encoded by the 40-kilobase cag pathogenicity island (cag PAI). The cag T4SS translocates the oncogenic bacterial protein CagA, as well as peptidoglycan, directly into the cytoplasm of gastric epithelial cells. One consequence of cag T4SS activity is NF-κB activation and increased production of pro-inflammatory cytokines such as IL-8.

When in contact with human gastric epithelial cells, H. pylori produce filamentous structures at the bacteria-host cell interface. Formation of these structures requires several cag PAI-encoded genes. These structures are thought to be an extracellular portion of the cag T4SS, analogous to the F pilus in conjugative T4SSs, and thus, these H. pylori structures have been termed cag T4SS pili. The composition of cag T4SS pili is not well defined, but several cag PAI-encoded proteins have been reported to localize to the pilus. The effector protein CagA has been localized at or near the tips of cag T4SS pili, and H. pylori mutants that fail to produce pili are unable to translocate CagA indicating that cag T4SS pili have an important role in T4SS function. In contrast to the requirement of pili for the actions of the H. pylori cag T4SS, the delivery of A. tumefaciens oncogenic T-DNA to target plant cells does not require biogenesis of the vir T4SS-associated T-pilus. Although the structures of related E. coli conjugative T4SSs have been resolved, the precise mechanism of DNA transport across the bacterial envelope remains under investigation.

### BRIEF DESCRIPTION OF THE DRAWINGS

Peptidomimetic small molecules disrupt activity of the H. pylori cag T4SS. (FIG. 1A) Compounds evaluated in this study contain a common peptidomimetic ring-fused 2-pyridone backbone structure (inset). (FIG. 1B) Effects of C10 and KSK85 on T4SS-dependent activation of IL-8 synthesis and secretion by cultured AGS gastric epithelial cells and (FIG. 1C) CagA translocation into cultured human gastric epithelial cells (150 μM final compound concentrations assayed). In FIG. 1C, densitometry analysis of tyrosine-phosphorylated CagA normalized to total CagA in 5 independent biological replicates was performed by ImageJ analysis. (FIG. 1D) Effects of C10 and KSK85 on H. pylori T4SS-dependent NF-κB activation in AGS cells. IL-8 secretion (FIG. 1E) and NF-κB activation (FIG. 1F) stimulated by recombinant human TNFα (in the absence of H. pylori). Graphs of IL-8 secretion and NF-κB activation depict the mean±SEM of at least 3 biological replicate experiments. P values in FIG. 1C were calculated by one-way ANOVA. See also FIG. 5 and Table 1.

KSK85 inhibits assembly of T4SS pili at the bacteria-host cell interface. Scanning electron microscopy (SEM) evaluating the T4SS piliation state of H. pylori (arrows) treated with vehicle (FIG. 2A), non-inhibitory compound GKP42 (FIG. 2B), C10 (FIG. 2C), or KSK85 (FIG. 2D). (FIG. 2E) Enumeration of T4SS pili per bacterial cell. (FIG. 2F) Proportion of H. pylori that elaborate T4SS pili in the presence of C10 and KSK85. Inset: The median number of pili per bacterial cell (boxes) and the maximum number of T4SS pili observed per individual H. pylori cell (whiskers). Scale bar equals 500 nm in panels FIGS. 2A-2D. Lines in FIG. 2E represent the geometric mean of each distribution.

C10 and KSK85 disrupt cag T4SS activity in the absence of CagA. (FIG. 3A) T4SS-dependent NF-κB activation by WT and H. pylori ΔcagA in the presence of C10 and (FIG. 3B) KSK85. (FIG. 3C) CFUs of adherent H. pylori on the surface of gastric epithelial cells at 6 h post-infection. Bars in FIG. 3A and FIG. 3B represent the standard error of the mean derived from four biological replicate experiments. Lines in FIG. 3C depict the geometric mean of 8 biological replicates. P values in FIG. 3C were calculated by two-tailed Mann-Whitney test.

Peptidomimetic small molecules target diverse type IV secretion systems. (FIG. 4A) DNA conjugation efficiency by the pKM101 and R1-16 encoded T4SSs in the presence of C10, KSK85, or GKP42 (150 μM assayed). Results represent mean conjugation efficiencies±SEM for five independent experiments. (FIG. 4B) A fluorescent-based assay was used to quantify μ-glucuronidase activity of N. benthamiana zones co-infiltrated with A. tumefaciens GV3101 pCAMBIA::GUS and DMSO, C10, KSK85, or GKP42 at the indicated concentrations. Bars depict the mean±SEM of three biological replicates containing at least three leaves with multiple zones of Agroinfiltration per leaf per biological replicate. P values were calculated by one-way ANOVA with Dunnett's post-hoc correction for multiple comparisons.

Effects of peptidomimetic small molecules on cell viability and T4SS-independent processes. (FIG. 5A) Schematic depicting the workflow of compound screening. A collection of ring-fused 2-pyridone compounds was initially evaluated at 150 μM final concentration for impact on host cell or H. pylori viability. Compounds that did not negatively affect either host or bacterial cell viability were subsequently evaluated for inhibition of H. pylori cag T4SS-dependent processes, including disruption of CagA translocation, induction of IL-8 secretion by cultured gastric epithelial cells, and NF-κK activation. (FIG. 5B) AGS and (FIG. 5C) H. pylori cell viability as determined by the level of cellular ATP content after 6 h exposure to compounds. (FIG. 5D) Total H. pylori adherence to gastric epithelial cell monolayers in the presence of ring-fused 2-pyridones (150 μM final concentration of all assayed compounds). (FIG. 5E) Representative image depicting CagA translocation into cultured gastric epithelial cells (tyrosine phosphorylated CagA, IB:α-PY99), versus levels of total CagA (IB: α-CagA). (FIG. 5F) Immunoblot depicting the relative amounts of H. pylori VacA secreted into cell culture supernatants when bacteria were grown in the presence of compounds or DMSO. (FIG. 5G) Effect of C10 and (FIG. 5H) KSK85 on cag T4SS-dependent activation of NF-κB in an AGS reporter cell line by multiple H. pylori strains. Bars in B-D represent the mean±SEM of at least 3 biological replicates. Data points in G and H depict the mean±SEM of at least 3 biological replicate experiments.

CagA is not required for T4SS pilus production or adherence to gastric epithelial cells. (FIG. 6A) Scanning electron microscopy analysis of T4SS pilus assembly by H. pylori ΔcagA. Scale bar represents 1 μm. (FIG. 6B) Total bacterial adherence of WT, ΔcagA, and ΔcagE to gastric epithelial cells in the presence of compounds. Bars represent the adherence of each strain normalized to DMSO treated samples (mean±standard error), and is representative of at least 2 biological replicate experiments.

Effects of peptidomimetic compounds on E. coli and A. tumefaciens viability. (FIG. 7A) Optical density measurements of E. coli MG1655 pKM101 growth in the presence of vehicle, C10, KSK85, or GKP42 measured at 1 h intervals. (FIG. 7B) A. tumefaciens cell viability as determined by the level of cellular ATP content after 24 h growth in compounds at the indicated concentrations. Data points in FIG. 7A represent the mean OD₆₀₀ of 6 independent samples. Data in FIG. 7B represent the mean cellular ATP content±SEM compared to DMSO vehicle control samples, and are representative of two biological replicate experiments.

Qualitative assessment of A. tumefaciens vir T4SS-dependent phenotypes. (FIG. 8A) Qualitative representation of T-DNA incorporation and GUS expression in tobacco leaves. Young, expanding leaves of Nicotiana benthamiana infiltrated with A. tumefaciens GV3101, or A. tumefaciens GV3101 harboring pCAMBIA 1305.2 intronic GUS reporter gene expression cassette (pCAMBIA::GUS). Representative image of N. benthamiana leaf stained histochemically for GUS enzyme activity demonstrating negative (GV3101) and positive (GV3101 pCAMBIA::GUS) incorporation of the β-glucuronidase reporter gene into N. benthamiana nuclear DNA. (FIG. 8B) Qualitative carrot disk tumor assay demonstrating marked reduction of A. tumefaciens C58-induced tumors after administration of a single administration (150 μM) of compound or equivalent volume of DMSO.

FIG. 9 shows the ability of synthetic small molecules to inhibit T4SS-dependent activation of human TLR9 signaling by H. pylori.

FIGS. 10A and 10B show the effects of test compounds to inhibit activation of TLR9 in HEK293 cells via H. pylori cag T4SS activity.

### SUMMARY OF THE INVENTION

In one aspect, disclosed are T4SSs inhibitors having formula (I),

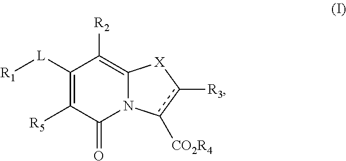

or a salt thereof, wherein

X is —O—, or —S—;

R₁ is bicyclic aryl or bicyclic heteroaryl;

L is —O—, —S—, alkylenyl, alkenylenyl, or heteroalkylenyl;

R₂ is cycloalkyl;

R₃ is hydrogen, halogen, or alkyl;

R₄ is hydrogen or alkyl;

R₅ is hydrogen, halogen, alkyl, amino, alkylamino, or dialkylamino; and

[symbol] is a single bond or a double bond;

wherein said bicyclic aryl, bicyclic heteroaryl, alkylenyl, alkenylenyl, heteroalkylenyl, cycloalkyl, and alkyl groups are each optionally substituted with one or more same or different substituents.

In another aspect, disclosed are T4SSs inhibitors having formula (I),

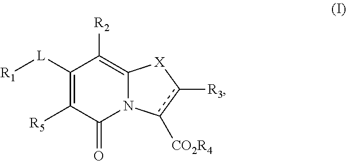

or a salt thereof, wherein

X is —O—, —S—, or —S(O)₂—;

R₁ is bicyclic aryl or bicyclic heteroaryl;

L is —O—, —S—, alkylenyl, alkenylenyl, or heteroalkylenyl;

R₂ is cycloalkyl;

R₃ is hydrogen, halogen, or alkyl;

R₄ is hydrogen or alkyl;

R₅ is hydrogen, halogen, alkyl, amino, alkylamino, or dialkylamino; and

[symbol] is a single bond or a double bond;

wherein said bicyclic aryl, bicyclic heteroaryl, alkylenyl, alkenylenyl, heteroalkylenyl, cycloalkyl, and alkyl groups are each optionally substituted with one or more same or different substituents.

Also disclosed are compositions comprising the compounds, methods of making the compounds, and methods of using the compounds and compositions for treating or preventing conditions or disorders caused by or associated with bacteria, including bacterial type IV secretion systems.

### DETAILED DESCRIPTION

Many human and plant pathogens utilize complex nanomachines called type IV secretion systems (T4SS) to transport protein and DNA to target cells. In addition to delivery of harmful effector proteins into target cells, T4SSs can disseminate genetic determinants that confer antibiotic resistance among bacterial populations. Disclosed herein are inhibitors of T4SSs, compositions including the inhibitors, and methods of using the inhibitors. The inhibitors can have formula (I), as described above. The compounds can inhibit the translocation of (i) protein, (ii) DNA, and (iii) peptidoglycan effector molecules in multiple, divergent bacterial species. The type IV secretion system inhibitors are able to disrupt transfer of multiple types of effector molecule cargo.

The T4SS targeting effects of the disclosed compounds include that of H. pylori, the inter-bacterial conjugative T4SS-mediated DNA transfer in E. coli, and impairment of vir T4SS-dependent T-DNA delivery by A. tumefaciens. In certain embodiments, the disclosed compounds (e.g., KSK85) impair formation of H. pylori cag T4SS-associated pili. In certain embodiments, the disclosed compounds (e.g., C10) impair T455-mediated transport without impacting T4SS pilus biogenesis. The disclosed compounds can prevent the spread of antibiotic resistance plasmids in E. coli populations, and diminish the transfer of tumor-inducing DNA from the plant pathogen A. tumefaciens to target cells.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain containing from 1 to 10 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tent-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkoxyalkyl” as used herein, refers to an alkoxy group, as defined herein, appended to the parent molecular moiety through an alkyl group, as defined herein.

The term “alkylene” or “alkylenyl”, as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “alkenyl” as used herein, refers to a linear or branched hydrocarbon chain having at least one carbon-carbon double bond, such as a vinyl group, an allyl group, an isopropenyl group, or the like.

The term “alkenylenyl”, as used herein, refers to a divalent group derived from a straight or branched chain hydrocarbon of 1 to 10 carbon atoms, having at least one carbon-carbon double bond.

The term “aryl” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, quinolinyl and tetrahydroquinolinyl.

The term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl.

The term “cycloalkenyl” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “fluoroalkyl” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “alkoxyfluoroalkyl” as used herein, refers to an alkoxy group, as defined herein, appended to the parent molecular moiety through a fluoroalkyl group, as defined herein.

The term “fluoroalkoxy” as used herein, means at least one fluoroalkyl group, as defined herein, is appended to the parent molecular moiety through an oxygen atom. Representative examples of fluoroalkyloxy include, but are not limited to, difluoromethoxy, trifluoromethoxy and 2,2,2-trifluoroethoxy.

The term “halogen” or “halo” as used herein, means Cl, Br, I, or F.

The term “haloalkyl” as used herein, means an alkyl group, as defined herein, in which one, two, three, four, five, six, seven or eight hydrogen atoms are replaced by a halogen.

The term “haloalkoxy” as used herein, means at least one haloalkyl group, as defined herein, is appended to the parent molecular moiety through an oxygen atom.

The term “heteroalkyl” as used herein, means an alkyl group, as defined herein, in which one or more of the carbon atoms has been replaced by a heteroatom independently selected from S, O, P and N. Representative examples of heteroalkyls include, but are not limited to, alkyl ethers, secondary and tertiary alkyl amines, amides, and alkyl sulfides.

The term “heteroalkylenyl”, as used herein, refers to a divalent group derived from a straight or branched chain hydrocarbon of 1 to 10 carbon atoms, in which one or more of the carbon atoms has been replaced by a heteroatom independently selected from S, O, P and N. Representative examples of heteroalkylenyl include, but are not limited to, —OCH₂—, —CH₂OCH₂—, —OCH₂OCH₂—, and —CH₂SCH₂CH₂CH₂—.

The term “heteroaryl” as used herein, refers to an aromatic monocyclic ring or an aromatic bicyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyridinyl (including pyridin-2-yl, pyridin-3-yl, pyridin-4-yl), pyrimidinyl, thiazolyl, and quinolinyl.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “hydroxyl” or “hydroxy” as used herein, means an —OH group.

In some instances, the number of carbon atoms in a hydrocarbyl substituent (e.g., alkyl or cycloalkyl) is indicated by the prefix “Cₓ-C_{y}-”, wherein x is the minimum and y is the maximum number of carbon atoms in the substituent. Thus, for example, “C₁-C₃-alkyl” refers to an alkyl substituent containing from 1 to 3 carbon atoms.

The term “substituted” refers to a group that may be further substituted with one or more non-hydrogen substituent groups. Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkyl sulfonyl, aryl sulfonyl, aminosulfonyl, sulfinyl, —COOH, ketone, amide, carbamate, and acyl.

For compounds described herein, groups and substituents thereof may be selected in accordance with permitted valence of the atoms and the sub stituents, such that the selections and substitutions result in a stable compound, e.g., which does not spontaneously undergo transformation such as by rearrangement, cyclization, elimination, etc.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOUNDS

In one aspect, disclosed are compounds of formula (I),

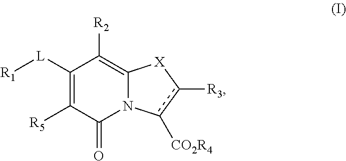

or a salt thereof, wherein

X is —O—, or —S—;

R₁ is bicyclic aryl or bicyclic heteroaryl;

L is —O—, —S—, alkylenyl, alkenylenyl, or heteroalkylenyl;

R₂ is cycloalkyl;

R₃ is hydrogen, halogen, or alkyl;

R₄ is hydrogen or alkyl;

R₅ is hydrogen, halogen, alkyl, amino, alkylamino, or dialkylamino; and

[symbol] is a single bond or a double bond;

wherein said bicyclic aryl, bicyclic heteroaryl, alkylenyl, alkenylenyl, heteroalkylenyl, cycloalkyl, and alkyl groups are each optionally substituted with one or more same or different substituents.

In another aspect, disclosed are T4SSs inhibitors having formula (I),

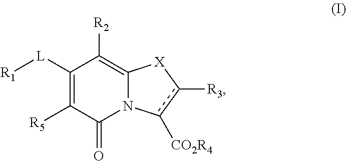

or a salt thereof, wherein

X is —O—, —S—, or —S—;

R₁ is bicyclic aryl or bicyclic heteroaryl;

L is —O—, —S—, alkylenyl, alkenylenyl, or heteroalkylenyl;

R₂ is cycloalkyl;

R₃ is hydrogen, halogen, or alkyl;

R₄ is hydrogen or alkyl;

R₅ is hydrogen, halogen, alkyl, amino, alkylamino, or dialkylamino; and

[symbol] is a single bond or a double bond;

wherein said bicyclic aryl, bicyclic heteroaryl, alkylenyl, alkenylenyl, heteroalkylenyl, cycloalkyl, and alkyl groups are each optionally substituted with one or more same or different substituents.

In certain embodiments, X is —O—. In certain embodiments, X is —S—. In certain embodiments, X is —S(O)—.

In certain embodiments, L is C₁-C₆-alkylenyl. In certain embodiments, L is —CH₂—.

In certain embodiments, R₁ is optionally substituted naphthalenyl. In certain embodiments, R₁ is a group of formula:

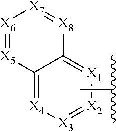

wherein X₁, X₂, X₃, X₄, X₅, X₆, X₇, and X₈ are each independently —CR₆ or N, wherein R₆, at each occurrence, is independently selected from hydrogen, halogen, C₁-C₆-alkyl, C₁-C₆-haloalkyl, C₁-C₆-alkoxy, and C₁-C₆-haloalkoxy. In certain embodiments, X₁, X₂, X₃, X₄, X₅, X₆, X₇, and X₈ are each independently —CR₆.

In certain embodiments, R₁ is optionally substituted naphthalenyl. In certain embodiments, R₁ is a group of formula:

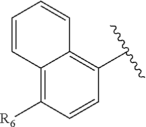

wherein R₆ is hydrogen, halogen, C₁-C₆-alkyl, C₁-C₆-haloalkyl, C₁-C₆-alkoxy, or C₁-C₆-haloalkoxy. In certain embodiments, R₆ is hydrogen. In certain embodiments, R₆ is C₁-C₆-alkoxy. In certain embodiments, R₆ is methoxy.

In certain embodiments, R₆ is hydrogen, halogen, C₁-C₆-alkyl, C₁-C₆-haloalkyl, C₁-C₆-alkoxy, C₁-C₆-haloalkoxy, or

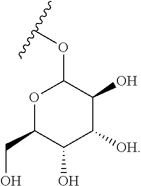 [SMILES: *OC1O[C@H](CO)[C@@H](O)[C@@H](O)[C@@H]1O]

In certain embodiments, R₆ is

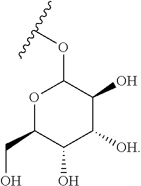 [SMILES: *OC1O[C@H](CO)[C@@H](O)[C@@H](O)[C@@H]1O]

In certain embodiments, R₂ is optionally substituted C₃-C₇-cycloalkyl. In certain embodiments, R₂ is cyclopropyl.

In certain embodiments, R₃ is hydrogen. In certain embodiments, R₃ is optionally substituted C₁-C₆-alkyl. In certain embodiments, R₃ is optionally substituted aryl-C₁-C₆-alkyl.

In certain embodiments, R₄ is hydrogen. In certain embodiments, R₄ is C₁-C₆-alkyl. In certain embodiments, R₄ is methyl.

In certain embodiments, R₅ is hydrogen.

In certain embodiments, the compound of formula (I) has formula (I-a),

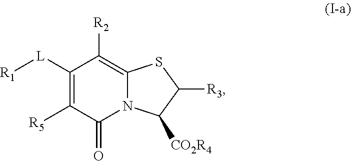

wherein R₁, R₂, R₃, R₄, R₅, and L are as defined above.

In certain embodiments, the compound of formula (I) has formula (I-b),

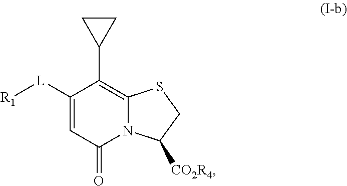

wherein R₁, R₄, and L are as defined above.

In certain embodiments, the compound of formula (I) has formula (I-c),

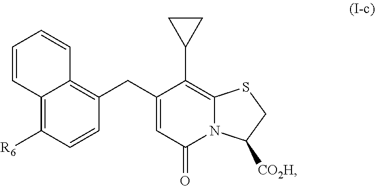

wherein R₆ is hydrogen, halogen, C₁-C₆-alkyl, C₁-C₆-haloalkyl, C₁-C₆-alkoxy, or C₁-C₆-haloalkoxy. In certain embodiments, R₆ is hydrogen. In certain embodiments, R₆ is C₁-C₆-alkoxy. In certain embodiments, R₆ is methoxy.

Representative compounds of formula (I) include, but are not limited to:

(3R)-7-[(4-methoxynaphthalen-1-yl)methyl]-8-cyclopropyl-5-oxo-2,3-dihydro-5H-[1,3]thiazolo[3,2-a]pyridine-3-carboxylic acid (KSK85);

(3R)-8-cyclopropyl-7-(naphthalen-1-ylmethyl)-5-oxo-3,5-dihydro-2H-thiazolo[3,2-a]pyridine-3-carboxylic acid (C10);

(S)-8-cyclopropyl-7-(naphthalen-1-ylmethyl)-5-oxo-2,3-dihydro-5H-thiazolo[3,2-a]pyridine-3-carboxylic acid (JG14);

8-cyclopropyl-7-(naphthalen-1-ylmethyl)-5-oxo-2,3-dihydro-5H-thiazolo[3,2-a]pyridine-3-carboxylic acid 1,1-dioxide (TW460);

(R)-8-cyclopropyl-7-((6-methoxynaphthalen-2-yl)methyl)-5-oxo-2,3-dihydro-5H-thiazolo[3,2-a]pyridine-3-carboxylic acid (CB158);

(R)-8-cyclopropyl-7-((4-ethoxynaphthalen-1-yl)methyl)-5-oxo-2,3-dihydro-5H-thiazolo[3,2-a]pyridine-3-carboxylic acid ((R)-JG131);

(S)-8-cyclopropyl-7-((4-methoxynaphthalen-1-yl)methyl)-5-oxo-2,3-dihydro-5H-thiazolo[3,2-a]pyridine-3-carboxylic acid ((S)-JG202);

(R)-8-cyclopropyl-7-(naphthalen-2-ylmethyl)-5-oxo-2,3-dihydro-5H-thiazolo[3,2-a]pyridine-3-carboxylic acid (KSK61);

8-cyclopropyl-5-oxo-7-((4-(trifluoromethyl)naphthalen-l1yl)methyl)-2,3-dihydro-5H-thiazolo[3,2-a]pyridine-3-carboxylic acid (KSK88); and

8-cyclopropyl-5-oxo-7-((4-(((3S,4R,5S,6R)-3,4,5-trihydroxy-6-(hydroxymethyl)tetrahydro-2H-pyran-2-yl)oxy)naphthalen-1-yl)methyl)-2,3-dihydro-5H-thiazolo[3,2-a]pyridine-3-carboxylic acid (PS402).

and salts thereof (e.g., pharmaceutically or agriculturally acceptable salts thereof).

In certain embodiments, (3R)-8-cyclopropyl-7-(naphthalen-1-ylmethyl)-5-oxo-3,5-dihydro-2H-thiazolo[3,2-a]pyridine-3-carboxylic acid (C10) is excluded from compounds of formula (I).

The disclosed compounds may exist as a stereoisomer when asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The disclosed compounds may exist as a salt, including pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 3. GENERAL SYNTHESIS

Compounds of formula (I) may be prepared by synthetic processes or by metabolic processes. Preparation of the compounds by metabolic processes includes those occurring in the human or animal body (in vivo) or processes occurring in vitro.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and T W Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 4. PHARMACEUTICAL COMPOSITION

The disclosed compounds may be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention [e.g., a compound of formula (I)] are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Dela. Suitable surfactants include those disclosed in the C.T.F.A. Cosmetic Ingredient Handbook, 1992, pp.587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound [e.g., a compound of formula (I)], and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### 5. AGRICULTURAL COMPOSITIONS

The disclosed compounds may be incorporated into agricultural compositions suitable for administration to a plant species. The compositions may be applied to plant propagating materials, including seeds and other regenerable plant parts, including cuttings, bulbs, rhizomes and tubers. The compositions may be applied to foliage or soil either prior to or following planting of plant propagating materials. Such applications may be made alone or in combination with fungicides, insecticides, nematicides and other agricultural agents.

As used herein the term “biologically effective amount” refers to that amount of a substance required to produce the desired effect on plant health, growth, or yield. Effective amounts of the composition will depend on several factors, including treatment method, plant species, propagating material type and environmental conditions.

The term “foliage” as used herein refers to the leaves of a plant.

Plant “growth” as used herein is defined by, but not limited to, measurements of seedling emergence, early growth, plant height, time to flowering, tillering (for grasses), days to maturity, vigor, biomass and yield.

The term “propagating material” means a seed or regenerable plant part. The term “regenerable plant part” means a part of the plant other than a seed from which a whole plant may be grown or regenerated when the plant part is placed in agricultural or horticultural growing media such as moistened soil, peat moss, sand, vermiculite, perlite, rock wool, fiberglass, coconut husk fiber, tree fern fiber, and the like, or even a completely liquid medium such as water. Regenerable plant parts commonly include rhizomes, tubers, bulbs and corms of such geophytic plant species as potato, sweet potato, yam, onion, dahlia, tulip, narcissus, etc. Regenerable plant parts include plant parts that are divided (e.g., cut) to preserve their ability to grow into a new plant. Therefore regenerable plant parts include viable divisions of rhizomes, tubers, bulbs and corms which retain meristematic tissue, such as an eye. Regenerable plant parts can also include other plant parts such as cut or separated stems and leaves from which some species of plants can be grown using horticultural or agricultural growing media. As referred to in the present disclosure and claims, unless otherwise indicated, the term “seed” includes both unsprouted seeds and seeds in which the testa (seed coat) still surrounds part of the emerging shoot and root. Foliage as defined in the present application includes all aerial plant organs, that is, the leaves, stems, flowers and fruit.

The term “rhizosphere” as defined herein refers to the area of soil that immediately surrounds and is affected by the plant's roots.

As used herein, the term “treating” in context of agricultural application means applying a biologically effective amount of a compound or agricultural composition, to a seed or other plant propagating material, plant foliage, or plant rhizosphere.

Agricultural compositions may contain effective amounts of active ingredient, diluent and surfactant.

Surfactants include, for example, ethoxylated alcohols, ethoxylated alkylphenols, ethoxylated sorbitan fatty acid esters, ethoxylated amines, ethoxylated fatty acids, esters and oils, dialkyl sulfosuccinates, alkyl sulfates, alkylaryl sulfonates, organosilicones, N,N-dialkyltaurates, glycol esters, phosphate esters, lignin sulfonates, naphthalene sulfonate formaldehyde condensates, polycarboxylates, and block polymers including polyoxyethylene/polyoxypropylene block copolymers.

Solid diluents include, for example, clays such as bentonite, montmorillonite, attapulgite and kaolin, starch, sugar, silica, talc, diatomaceous earth, urea, calcium carbonate, sodium carbonate and bicarbonate, and sodium sulfate. Liquid diluents include, for example, water, N,N-dimethylformamide, dimethyl sulfoxide, N-alkylpyrrolidone, ethylene glycol, polypropylene glycol, propylene carbonate, dibasic esters, paraffins, alkylbenzenes, alkylnaphthalenes, oils of olive, castor, linseed, tung, sesame, corn, peanut, cotton-seed, soybean, rape-seed and coconut, fatty acid esters, ketones such as cyclohexanone, 2-heptanone, isophorone and 4-hydroxy-4-methyl-2-pentanone, and alcohols such as methanol, cyclohexanol, decanol, benzyl and tetrahydrofurfuryl alcohol.

Solutions, including emulsifiable concentrates, can be prepared by simply mixing the ingredients. Dusts and powders can be prepared by blending and, usually, grinding as in a hammer mill or fluid-energy mill. Suspensions can be prepared by wet-milling. Granules and pellets can be prepared by spraying the active material upon preformed granular carriers or by agglomeration techniques.

The compositions used for treating propagating materials, or plants grown therefrom, can include an effective amount of one or more other biologically active compounds or agents. Suitable additional compounds or agents include, but are not limited to, insecticides, fungicides, nematocides, bactericides, acaricides, entomopathogenic bacteria, viruses or fungi, growth regulators such as rooting stimulants, chemosterilants, repellents, attractants, pheromones, feeding stimulants and other signal compounds including apocarotenoids, flavonoids, jasmonates and strigolactones. These compounds can also be formulated into mixtures or multi-component formulations.

Examples of such biologically active compounds or agents with which compounds disclosed herein can be mixed or formulated are: insecticides such as abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron; fungicides such as acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (SSF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin; nematocides such as aldicarb, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including Bacillus thuringiensis (including ssp. aizawai and kurstaki), Bacillus thuringiensis delta-endotoxin, baculoviruses, and entomopathogenic bacteria, viruses and fungi.

Water-dispersible or soluble powders, granules, tablets, emulsifiable concentrates, aqueous suspension concentrates and the like are formulations suitable for aqueous drenches of growing media. Drenches are most satisfactory for treating growing media that have relatively high porosity, such as light soils or artificial growing medium comprising porous materials such as peat moss, perlite, vermiculite and the like. The drench liquid including a disclosed compound or agricultural composition can also be added to a liquid growing medium (i.e. hydroponics), which causes the compound to become part of the liquid growing medium. The biologically effective amount will vary with several factors including, but not limited to, plant species, propagating material type and environmental conditions. The concentration of a disclosed compound in the drench liquid may range between about 10⁻⁻⁵M to 10⁻¹²M of the composition, more typically between about 10⁻⁶M to 10⁻¹⁰M.

The compounds and compositions can also be applied by mixing it as a dry powder or granule formulation with the growing medium. Because this method of application does not require first dispersing or dissolving in water, the dry powder or granule formulations need not be highly dispersible or soluble. While in a nursery box the entire body of growing medium may be treated, in an agricultural field only the soil in the vicinity of the propagating material is typically treated for environmental and cost reasons. To minimize application effort and expense, a composition can be most efficiently applied concurrently with propagating material planting (e.g., seeding). For in-furrow application, the compositions can be applied directly behind the planter shoe. For T-band application, the compositions can be applied in a band over the row behind the planter shoe and behind or usually in front of the press wheel. The biologically effective amount will vary with several factors including, but not limited to, plant species, propagating material type and environmental conditions. The concentration of compound in the growing medium locus can generally be between about 10⁻⁵M to 10⁻¹²M of the composition, more typically between about 10⁻⁶M to 10⁻¹⁰M.

A propagating material can be directly treated by soaking it in a solution or dispersion of a disclosed compound or composition. Although this application method is useful for propagating materials of all types, treatment of large seeds (e.g., having a mean diameter of at least 3 mm) may be more effective than treatment of small seeds for providing efficacy. Treatment of propagating materials such as tubers, bulbs, corms, rhizomes and stem and leaf cuttings also can provide effective treatment of the developing plant in addition to the propagating material. The compositions useful for growing-medium drenches are generally also useful for soaking treatments. The soaking medium comprises a nonphytotoxic liquid, generally water-based although it may contain nonphytotoxic amounts of other solvents such as methanol, ethanol, isopropanol, ethylene glycol, propylene glycol, propylene carbonate, benzyl alcohol, dibasic esters, acetone, methyl acetate, ethyl acetate, cyclohexanone, dimethylsulfoxide and N-methylpyrrolidone, which may be useful for enhancing solubility of the compound and penetration into the propagating material. A surfactant can facilitate wetting of the propagating material and penetration of the compound. The biologically effective amount will vary with several factors including, but not limited to, plant species, propagating material type and environmental conditions. The concentration of compound in the soaking liquid is generally between about 10⁻⁵M to 10⁻¹²M of the composition, more typically between about 10⁻⁶M to 10⁻¹⁰M. One skilled in the art can easily determine the biologically effective concentration necessary for the desired level of efficacy. The soaking time can vary from one minute to one day or even longer. Indeed, the propagating material can remain in the treatment liquid while it is germinating or sprouting (e.g., sprouting of rice seeds prior to direct seeding). As shoot and root emerge through the testa (seed coat), the shoot and root directly contact the solution comprising the compound. For treatment of sprouting seeds of large-seeded crops such as rice, treatment times of about 8 to 48 hours, e.g., about 24 hours, is typical. Shorter times are most useful for treating small seeds.

A propagating material can also be coated with a composition comprising a biologically effective amount of a compound. The coatings are capable of effecting a slow release of a compound by diffusion into the propagating material and surrounding medium. Coatings include dry dusts or powders adhering to the propagating material by action of a sticking agent such as methylcellulose or gum arabic. Coatings can also be prepared from suspension concentrates, water-dispersible powders or emulsions that are suspended in water, sprayed on the propagating material in a tumbling device and then dried. Compounds that are dissolved in the solvent can be sprayed on the tumbling propagating material and the solvent then evaporated. Such compositions can include ingredients promoting adhesion of the coating to the propagating material. The compositions may also contain surfactants promoting wetting of the propagating material. Generally water is used, but other volatile solvents with low phytotoxicity such as methanol, ethanol, methyl acetate, ethyl acetate, acetone, etc. may be employed alone or in combination. Volatile solvents are those with a normal boiling point less than about 100° C. Drying can be conducted in a way not to injure the propagating material or induce premature germination or sprouting.

The thickness of coatings can vary from adhering dusts to thin films to pellet layers about 0.5 to 5 mm thick. Propagating material coatings can comprise more than one adhering layer. Generally pellets are most satisfactory for small seeds, because their ability to provide a biologically effective amount of an active agent is not limited by the surface area of the seed, and pelleting small seeds also facilitates seed transfer and planting operations. Because of their larger size and surface area, large seeds and bulbs, tubers, corms and rhizomes and their viable cuttings are generally not pelleted, but instead coated with powders or thin films.

Propagating materials contacted with compounds disclosed herein include seeds. Suitable seeds include seeds of wheat, durum wheat, barley, oat, rye, maize, sorghum, rice, wild rice, cotton, flax, sunflower, soybean, garden bean, lima bean, broad bean, garden pea, peanut, alfalfa, beet, garden lettuce, rapeseed, cole crop, turnip, leaf mustard, black mustard, tomato, potato, pepper, eggplant, tobacco, cucumber, muskmelon, watermelon, squash, carrot, zinnia, cosmos, chrysanthemum, sweet scabious, snapdragon, gerbera, babys-breath, statice, blazing star, lisianthus, yarrow, marigold, pansy, impatiens, petunia, geranium and coleus. Of note are seeds of cotton, maize, soybean and rice. Propagating materials contacted with compounds in accordance with the disclosure also include rhizomes, tubers, bulbs or corms, or viable divisions thereof. Suitable rhizomes, tubers, bulbs and corms, or viable divisions thereof include those of potato, sweet potato, yam, garden onion, tulip, gladiolus, lily, narcissus, dahlia, iris, crocus, anemone, hyacinth, grape-hyacinth, freesia, ornamental onion, wood-sorrel, squill, cyclamen, glory-of-the-snow, striped squill, calla lily, gloxinia and tuberous begonia. Of note are rhizomes, tubers, bulbs and corms, or viable division thereof of potato, sweet potato, garden onion, tulip, daffodil, crocus and hyacinth. Propagating materials contacted with the disclosed compounds can also include stems and leaf cuttings.

One embodiment of a propagating material contacted with a disclosed compound is a propagating material coated with a composition comprising a disclosed compound and a film former or adhesive agent. Compositions which comprise a biologically effective amount of a disclosed compound and a film former or adhesive agent, can further comprise an effective amount of at least one additional biologically active compound or agent. Of note are compositions comprising an arthropodicide of the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics. Also of note are compositions comprising at least one additional biologically active compound or agent selected from the group consisting of abamectin, acephate, acetamiprid, amidoflumet (S-1955), avermectin, azadirachtin, azinphos-methyl, bifenthrin, binfenazate, buprofezin, carbofuran, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, diflubenzuron, dimethoate, diofenolan, emamectin, endosulfan, esfenvalerate, ethiprole, fenothicarb, fenoxycarb, fenpropathrin, fenproximate, fenvalerate, fipronil, flonicamid, flucythrinate, tau-fluvalinate, flufenerim (UR-50701), flufenoxuron, fonophos, halofenozide, hexaflumuron, imidacloprid, indoxacarb, isofenphos, lufenuron, malathion, metaldehyde, methamidophos, methidathion, methomyl, methoprene, methoxychlor, monocrotophos, methoxyfenozide, nithiazin, novaluron, noviflumuron (XDE-007), oxamyl, parathion, parathion-methyl, permethrin, phorate, phosalone, phosmet, phosphamidon, pirimicarb, profenofos, pymetrozine, pyridalyl, pyriproxyfen, rotenone, spinosad, spiromesifin (BSN 2060), sulprofos, tebufenozide, teflubenzuron, tefluthrin, terbufos, tetrachlorvinphos, thiacloprid, thiamethoxam, thiodicarb, thiosultap-sodium, tralomethrin, trichlorfon and triflumuron, aldicarb, oxamyl, fenamiphos, amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad; and biological agents such as Bacillus thuringiensis (including ssp. aizawai and kurstaki), Bacillus thuringiensis delta-endotoxin, baculoviruses, and entomopathogenic bacteria, viruses and fungi. Also of note are compositions comprising at least one additional biologically active compound or agent selected from fungicides of the group consisting of acibenzolar, azoxystrobin, benomyl, blasticidin-S, Bordeaux mixture (tribasic copper sulfate), bromuconazole, carpropamid, captafol, captan, carbendazim, chloroneb, chlorothalonil, copper oxychloride, copper salts, cyflufenamid, cymoxanil, cyproconazole, cyprodinil, (S)-3,5-dichloro-N-(3-chloro-1-ethyl-1-methyl-2-oxopropyl)-4-methylbenzamide (RH 7281), diclocymet (S-2900), diclomezine, dicloran, difenoconazole, (S)-3,5-dihydro-5-methyl-2-(methylthio)-5-phenyl-3-(phenylamino)-4H-imidazol-4-one (RP 407213), dimethomorph, dimoxystrobin, diniconazole, diniconazole-M, dodine, edifenphos, epoxiconazole, famoxadone, fenamidone, fenarimol, fenbuconazole, fencaramid (SZX0722), fenpiclonil, fenpropidin, fenpropimorph, fentin acetate, fentin hydroxide, fluazinam, fludioxonil, flumetover (RPA 403397), flumorf/flumorlin (SYP-L190), fluoxastrobin (HEC 5725), fluquinconazole, flusilazole, flutolanil, flutriafol, folpet, fosetyl-aluminum, furalaxyl, furametapyr (S-82658), hexaconazole, ipconazole, iprobenfos, iprodione, isoprothiolane, kasugamycin, kresoxim-methyl, mancozeb, maneb, mefenoxam, mepronil, metalaxyl, metconazole, metominostrobin/fenominostrobin (S SF-126), metrafenone (AC 375839), myclobutanil, neo-asozin (ferric methanearsonate), nicobifen (BAS 510), orysastrobin, oxadixyl, penconazole, pencycuron, probenazole, prochloraz, propamocarb, propiconazole, proquinazid (DPX-KQ926), prothioconazole (JAU 6476), pyrifenox, pyraclostrobin, pyrimethanil, pyroquilon, quinoxyfen, spiroxamine, sulfur, tebuconazole, tetraconazole, thiabendazole, thifluzamide, thiophanate-methyl, thiram, tiadinil, triadimefon, triadimenol, tricyclazole, trifloxystrobin, triticonazole, validamycin and vinclozolin.

Generally, a propagating material coating includes a disclosed compound, and a film former or sticking agent. The coating may further comprise formulation aids such as a dispersant, a surfactant, a carrier and optionally an antifoam and dye. The biologically effective amount will vary with several factors including, but not limited to, plant species, propagating material type and environmental conditions. The coating prefereably does not inhibit germination or sprouting of the propagating material.

The film former or adhesive agent component of the propagating material coating can be composed of an adhesive polymer that may be natural or synthetic and is without phytotoxic effect on the propagating material to be coated. The film former or sticking agent may be selected from polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxymethylcelluloses, hydroxy-propylcellulose, hydroxymethylpropylcelluloses, polyvinylpyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof. The amount of film former or sticking agent in the formulation is generally in the range of about 0.001 to 100% of the weight of the propagating material. For large seeds the amount of film former or sticking agent is typically in the range of about 0.05 to 5% of the seed weight; for small seeds the amount is typically in the range of about 1 to 100%, but can be greater than 100% of seed weight in pelleting. For other propagating materials the amount of film former or sticking agent is typically in the range of 0.001 to 2% of the propagating material weight.

Formulation aids assist in the production or process of propagating material treatment and include, but are not limited, to dispersants, surfactants, carriers, antifoams and dyes. Useful dispersants can include highly water-soluble anionic surfactants like Borresperse™ CA, Morwet® D425 and the like. Useful surfactants can include highly water-soluble nonionic surfactants like Pluronic® F108, Brij® 78 and the like. Useful carriers can include liquids like water and oils which are water-soluble such as alcohols. Useful carriers can also include fillers like woodflours, clays, activated carbon, diatomaceous earth, fine-grain inorganic solids, calcium carbonate and the like. Clays and inorganic solids which may be used include calcium bentonite, kaolin, china clay, talc, perlite, mica, vermiculite, silicas, quartz powder, montmorillonite and mixtures thereof. Antifoams can include water dispersible liquids comprising polyorganic siloxanes like Rhodorsil® 416. Dyes can include water dispersible liquid colorant compositions like Pro-Ized® Colorant Red. The amount of formulation aids used may vary, but generally the weight of the components will be in the range of about 0.001 to 10000% of the propagating material weight, with the percentages above 100% being mainly used for pelleting small seed. For nonpelleted seed generally the amount of formulating aids is about 0.01 to 45% of the seed weight and typically about 0.1 to 15% of the seed weight. For propagating materials other than seeds, the amount of formulation aids generally is about 0.001 to 10% of the propagating material weight.

Dusts or powders may be applied by tumbling the propagating material with a formulation and a sticking agent to cause the dust or powder to adhere to the propagating material and not fall off during packaging or transportation. Dusts or powders can also be applied by adding the dust or powder directly to the tumbling bed of propagating materials, followed by spraying a carrier liquid onto the seed and drying. Dusts and powders can also be applied by treating (e.g., dipping) at least a portion of the propagating material with a solvent such as water, optionally comprising a sticking agent, and dipping the treated portion into a supply of the dry dust or powder. This method can be particularly useful for coating stem cuttings. Propagating materials can also be dipped into compositions of wetted powders, solutions, suspoemulsions, emulfiable concentrates and emulsions in water, and then dried or directly planted in the growing medium.

Propagating materials may also be coated by spraying a suspension concentrate directly into a tumbling bed of propagating materials and then drying the propagating materials. Alternatively, other formulation types like wetted powders, solutions, suspoemulsions, emulsifiable concentrates and emulsions in water may be sprayed on the propagating materials. This process is particularly useful for applying film coatings to seeds.

For coating seed, the seed and coating material are mixed in any variety of conventional seed coating apparatus. The rate of rolling and coating application depends upon the seed. For large oblong seeds such as those of cotton, a satisfactory seed coating apparatus comprises a rotating type pan with lifting vanes turned at sufficient rpm to maintain a rolling action of the seed, facilitating uniform coverage. For seed coating formulations applied as liquids, the seed coating must be applied over sufficient time to allow drying to minimize clumping of the seed. Using forced air or heated forced air can facilitate an increased rate of application. The process may be a batch or continuous process. A continuous process allows the seeds to flow continuously throughout the product run. New seeds enter the pan in a steady stream to replace coated seeds exiting the pan.

The seed coating process is not limited to thin film coating and may also include seed pelleting. The pelleting process typically increases the seed weight from 2 to 100 times and can be used to also improve the shape of the seed for use in mechanical seeders. Pelleting compositions generally contain a solid diluent, which is typically an insoluble particulate material, such as clay, ground limestone, powdered silica, etc., to provide bulk in addition to a binder such as an artificial polymer (e.g., polyvinyl alcohol, hydrolyzed polyvinyl acetates, polyvinyl methyl ether, polyvinyl methyl ether-maleic anhydride copolymer, and polyvinylpyrrolidinone) or natural polymer (e.g., alginates, karaya gum, jaguar gum, tragacanth gum, polysaccharide gum, mucilage). After sufficient layers have been built up, the coat is dried and the pellets graded.

Seed varieties and seeds with specific transgenic traits may be tested to determine which seed treatment options and application rates may complement such varieties and transgenic traits in order to enhance yield.

In another embodiment, the disclosed compounds and compositions can be applied as a foliar formulation. Such formulations will generally include at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake. Effective foliar formulations will typically contain from about 10⁻⁵M to 10⁻¹²M of the composition.

In another embodiment, the compounds and compositions can be applied to soil either prior to or following planting of plant propagating materials. Compositions can be applied as a soil drench of a liquid formulation, a granular formulation to the soil, a nursery box treatment or a dip of transplants. Other methods of contact include application of a compound or a composition by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. Effective soil formulations will typically contain from about 10⁻⁵M to 10⁻¹²M of the composition.

The disclosed compounds and compositions can be applied to virtually all plant species. Seeds that can be treated include, for example, wheat (Triticum aestivum L.), durum wheat (Triticum durum Desf.), barley (Hordeum vulgare L.), oat (Avena sativa L.), rye (Secale cereale L.), maize (Zea mays L.), sorghum (Sorghum vulgare Pers.), rice (Oryza sativa L.), wild rice (Zizania aquatica L.), millet (Eleusine coracana, Panicum miliaceum), cotton (Gossypium barbadense L. and G. hirsutum L.), flax (Linum usitatissimum L.), sunflower (Helianthus annuus L.), soybean (Glycine max Merr.), garden bean (Phaseolus vulgaris L.), lima bean (Phaseolus limensis Macf.), broad bean (Vicia faba L.), garden pea (Pisum sativum L.), peanut (Arachis hypogaea L.), alfalfa (Medicago sativa L.), beet (Beta vulgaris L.), garden lettuce (Lactuca sativa L.), rapeseed (Brassica rapa L. and B. napus L.), cole crops such as cabbage, cauliflower and broccoli (Brassica oleracea L.), turnip (Brassica rapa L.), leaf (oriental) mustard (Brassica juncea Coss.), black mustard (Brassica nigra Koch), tomato (Lycopersicon esculentum Mill.), potato (Solanum tuberosum L.), pepper (Capsicum frutescens L.), eggplant (Solanum melongena L.), tobacco (Nicotiana tabacum), cucumber (Cucumis sativus L.), muskmelon (Cucumis melo L.), watermelon (Citrullus vulgaris Schrad.), squash (Curcurbita pepo L., C. moschata Duchesne. and C. maxima Duchesne.), carrot (Daucus carota L.), zinnia (Zinnia elegans Jacq.), cosmos (e.g., Cosmos bipinnatus Cay.), chrysanthemum (Chrysanthemum spp.), sweet scabious (Scabiosa atropurpurea L.), snapdragon (Antirrhinum majus L.), gerbera (Gerbera jamesonii Bolus), babys-breath (Gypsophila paniculata L., G. repens L. and G. elegans Bieb.), statice (e.g., Limonium sinuatum Mill., L. sinense Kuntze.), blazing star (e.g., Liatris spicata Willd., L. pycnostachya Michx., L. scariosa Willd.), lisianthus (e.g., Eustoma grandiflorum (Raf.) Shinn), yarrow (e.g., Achillea filipendulina Lam., A. millefolium L.), marigold (e.g., Tagetes patula L., T erecta L.), pansy (e.g., Viola cornuta L., V. tricolor L.), impatiens (e.g., Impatiens balsamina L.) petunia (Petunia spp.), geranium (Geranium spp.) and coleus (e.g., Solenostemon scutellarioides (L.) Codd). Not only seeds, but also rhizomes, tubers, bulbs or corms, including viable cuttings thereof, can be treated from, for example, potato (Solanum tuberosum L.), sweet potato (Ipomoea batatas L.), yam (Dioscorea cayenensis Lam. and D. rotundata Poir.), garden onion (e.g., Allium cepa L.), tulip (Tulipa spp.), gladiolus (Gladiolus spp.), lily (Lilium spp.), narcissus (Narcissus spp.), dahlia (e.g., Dahlia pinnata Cay.), iris (Iris germanica L. and other species), crocus (Crocus spp.), anemone (Anemone spp.), hyacinth (Hyacinth spp.), grape-hyacinth (Muscari spp.), freesia (e.g., Freesia refracta Klatt., F. armstrongii W. Wats), ornamental onion (Allium spp.), wood-sorrel (Oxalis spp.), squill (Scilla peruviana L. and other species), cyclamen (Cyclamen persicum Mill. and other species), glory-of-the-snow (Chionodoxa luciliae Boiss. and other species), striped squill (Puschkinia scilloides Adams), calla lily (Zantedeschia aethiopica Spreng., Z. elliottiana Engler and other species), gloxinia (Sinnigia speciosa Benth. & Hook.) and tuberous begonia (Begonia tuberhybrida Voss.). Stem cuttings can be treated, including those from such plants as sugarcane (Saccharum officinarum L.), carnation (Dianthus caryophyllus L.), florists chrysanthemum (Chrysanthemum mortifolium Ramat.), begonia (Begonia spp.), geranium (Geranium spp.), coleus (e.g., Solenostemon scutellarioides (L.) Codd) and poinsettia (Euphorbia pulcherrima Willd.). Leaf cuttings which can be treated include those from begonia (Begonia spp.), african-violet (e.g., Saintpaulia ionantha Wendl.) and sedum (Sedum spp.).

### 6. METHODS

The disclosed compounds and compositions may be used in methods for inhibiting the biogenesis and function of bacterial type IV secretion systems. The disclosed compounds and compositions may be used in methods for inhibiting the transfer of macromolecular substrates (e.g., proteins, DNA) from bacterium to bacterium, or from bacterium to host. The disclosed compounds and compositions may be used in methods for inhibiting transfer of antibiotic resistance from bacterium to bacterium. The methods may comprise administering to a subject (e.g., an animal such as a mammal, or a plant) in need of such treatment a compound or composition comprising a therapeutically effective amount of the compound of formula (I).

The disclosed compounds and compositions may be used in methods for treatment of Helicobacter pylori infections. H. pylori is the causative agent of severe gastric disease, including peptic ulcers and gastric adenocarcinoma, and colonization by H. pylori that harbor the cag type IV secretion system (inhibited by our molecules) is associated with a significantly increased risk of gastric cancer. H. pylori that possess the cag type IV secretion system are able to inject the bacterial oncoprotein CagA into host cells, thus augmenting carcinogenesis.

The disclosed compounds and compositions may be used in methods for treating or preventing conditions and disorders caused by or associated with Helicobacter pylori infections, including gastric diseases and conditions, such as for example, peptic ulcers, gastric adenocarcinoma, gastric cancer, gastritis, gastric ulcer, duodenal ulcer, non-ulcer dyspepsia syndrome, gastric MALT lymphoma, gastric hyperplastic polyp, digestive system cancer or pancreatitis resulting from hypergastrinemia caused by Helicobacter pylori, and an inflammatory bowel disease caused by Helicobacter pylori.

The disclosed compounds and compositions may be used in methods for treatment of Agrobacterium tumefaciens infections. A. tumefaciens uses the vir type IV secretion system to transfer tumor-inducing DNA into host plant cells and co-opts the host cellular machinery to develop a replicative niche that leads to development of crown gall tumors and plant disease. The disclosed compounds and compositions may be used in methods for treating or preventing conditions or disorders caused by or associated with Agrobacterium tumefaciens infections, including crown gall tumors and plant disease.

The disclosed compounds and compositions may be used in methods for preventing or inhibiting transfer of antibiotic resistance among strains of bacteria, including type IV secretion system-mediated gene exchange.

### 7. EXAMPLES

The compounds and processes will be better understood by reference to the following examples, which are intended as an illustration of and not a limitation upon the scope of the invention.

All reagents and solvents were used as received from commercial suppliers, unless indicated otherwise. Triethylamine was passed through activated alumina oxide and dried over 3Å molecular sieves prior to use. Microwave reactions were performed using a Biotage Initiator microwave synthesizer in sealed vessels with temperature monitoring by an internal IR probe. TLC was performed on aluminum backed silica gel plates (median pore size 60Å) and detected with UV light at 254 nm. Column chromatography was performed using silica gel with average particle diameter 50 μM (range 40-65 μM, pore diameter 53Å) and eluents are given in brackets. HPLC purifications were performed on a system equipped with a 250×21.5 mm Nucleodur® C18 HTEC (particle size 5 μM) semi-preparatory column using a flow rate of 20 mL/min and detection at 220 nm. Optical rotation was measured with a polarimeter at 25° C. at 589 nm. ¹H and ¹³C NMR spectra were recorded on a 400 or 600 MHz spectrometer at 298 K and calibrated by using the residual peak of the solvent as the internal standard (CDCl₃: δ_{H}=7.26 ppm; δ_{C}=77.16 ppm; CD₃OD: δ_{H}=3.31 ppm; δ_{C}=49.00 ppm; DMSO-d₆: δ_{H}=2.50 ppm; δ_{C}=39.50 ppm). HRMS was performed using a mass spectrometer with ESI-TOF (ESI+) with sodium formate used as the calibration chemical. Compounds are named according to IUPAC nomenclature by ACD ChemSketch 12.01 (Windows, Advanced Chemistry Development, Toronto, Canada).

C10 was prepared as described previously (Chorell E, Pinkner J S, Phan G, Edvinsson S, Buelens F, Remaut H, Waksman G, Hultgren S J, Almqvist F. 2010. Design and synthesis of C-2 substituted thiazolo and dihydrothiazolo ring-fused 2-pyridones: pilicides with increased antivirulence activity. Journal of Medicinal Chemistry 53:5690-5695). The chloromethyl intermediate S3 and the methyl ester S5 were prepared as described previously (Sellstedt M, Prasad G K, Krishnan S K, Almqvist F. 2012. Directed diversity-oriented synthesis. Ring-fused 5- to 10-membered rings from a common peptidomimetic 2-pyridone precursor. Tetrahedron Letters 53:6022-6024). 1-Bromo-4-methoxynaphthalene (S1) was prepared according to published procedures (Carreno M C, Garcia Ruano J L, Sanz G, Toledo M A, Urbano A. 1995. N-bromosuccinimide in acetonitrile: A mild and regiospecific nuclear brominating reagent for methoxybenzenes and naphthalenes. The Journal of Organic Chemistry 60:5328-5331).

A. Synthetic Procedures and Characterization of Compounds

### Example 1

### (3R)-7-[(4-methoxynaphthalen-1-yl)methyl]-8-cyclopropyl-5-oxo-2,3-dihydro-5H-[1,3]thiazolo[3,2-a]pyridine-3-carboxylate (KSK85)

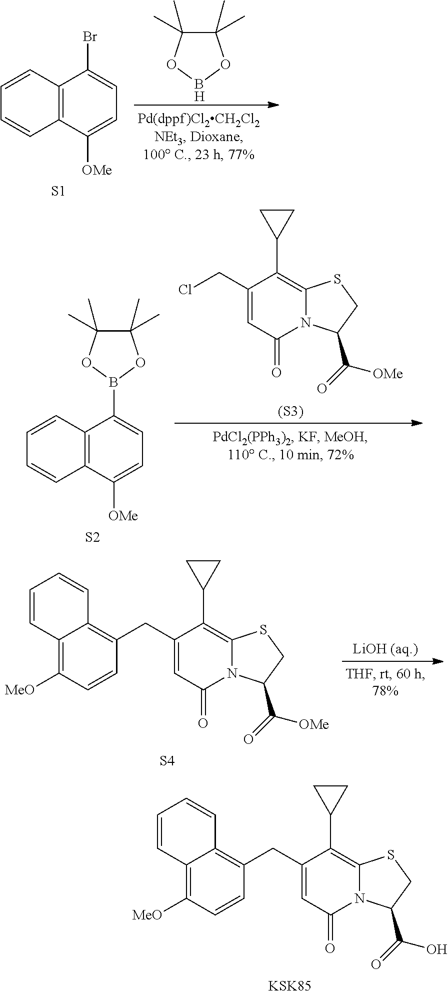

2-(4-Methoxy-1-naphthyl)-4,4,5,5-tetramethyl-1,3,2-dioxaborolane (S2). 4,4,5,5-Tetramethyl-1,3,2-dioxaborolane (1.92 mL, 15.00 mmol) was added dropwise to a solution of 1-bromo-4-methoxynaphthalene S1 (1.19 g, 5.00 mmol), NEt₃ (2.79 mL, 20 mmol) and Pd(ddpf)Cl₂.CH₂Cl₂ (510 mg, 0.625 mmol) in anhydrous dioxane (15 mL) and the reaction mixture heated at 100° C. for 23 h. After cooling to room temperature, the reaction was quenched cautiously with water (˜20 mL) and extracted with CH₂Cl₂ (3×35 mL). The combined organic extracts were washed with brine (50 mL), dried (Na₂SO₄) and concentrated under reduced pressure. Purification by flash chromatography (SiO₂; EtOAc/heptane; 0-30%) afforded the pinacol ester as an off-white solid (1.09 g, 77%). ¹H NMR (400 MHz, CDCl₃) δ=1.41 (s, 12H), 4.02 (s, 3H), 6.82 (d, J=7.8 Hz, 1H), 7.43-7.48 (m, 1H), 7.53-7.57 (m, 1H), 8.04 (d, J=7.8 Hz, 1H), 8.28 (dd, J=0.9, 8.3 Hz, 1H), 8.75 (d, J=8.3 Hz, 1H). ¹³C NMR (100 MHz, CDCl₃) δ=25.1, 55.6, 83.5, 103.3, 122.1, 125.0, 125.5, 127.0, 128.3, 137.0, 138.3, 158.3. HRMS (ESI+) (m/z): [M+H]⁺ calcd. for C₁₇H₂₂BO₃, 285.1657; found, 285.1643.

Methyl (3R)-7-[(4-methoxynaphthalen-1-yl)methyl]-8-cyclopropyl-5-oxo-2,3-dihydro-5H-[1,3]thiazolo[3,2-a]pyridine-3-carboxylate (S4). The chloromethyl analogue S3 (150 mg, 0.500 mmol), boronic acid pinacol ester S2 (284.2 mg, 1.00 mmol), PdCl₂(PPh₃)₂ (35 mg, 0.005 mmol) and KF (58 mg, 1.00 mmol) were dissolved in anhydrous MeOH (8 mL) and heated by MWI at 110° C. for 10 min. The reaction mixture was quenched with saturated aqueous NaHCO₃ solution (15 mL) and extracted with CH₂Cl₂ (3×20 mL). The combined organic extracts were washed successively with water and brine (50 mL each), dried (Na₂SO₄) and concentrated under reduced pressure. The crude residue was dissolved in EtOAc and filtered through Celite®, washing with EtOAc, and again concentrated under reduced pressure. Purification by flash chromatography (SiO₂; EtOAc/heptane; 20-100%) afforded the product as a pale brown oil (151 mg, 72%). ¹H NMR (400 MHz, CDCl₃) δ=0.69-0.78 (m, 2H), 0.87-1.03 (m, 2H), 1.61-1.71 (m, 1H), 3.50 (dd, J=2.3, 11.8 Hz, 1H), 3.66 (dd, J=8.5, 11.7 Hz, 1H), 3.78 (s, 3H), 4.00 (s, 3H), 4.25 (d, J=17.3 Hz, 1H), 4.41 (d, J=17.3 Hz, 1H), 5.56 (dd, J=2.2, 8.5 Hz, 1H), 5.75 (s, 1H), 6.75 (d, J=7.8 Hz, 1H), 7.17 (d, J=7.8 Hz, 1H), 7.44-7.50 (m, 2H), 7.68-7.74 (m, 1H), 8.28-8.33 (m, 1H). ¹³C NMR (100 MHz, CDCl₃) δ=7.6, 7.9, 11.3, 31.9, 36.0, 53.4, 55.7, 62.8, 103.5, 113.9, 115.4, 122.9, 123.8, 125.2, 125.9, 126.2, 126.8, 127.7, 132.8, 146.9, 155.1, 157.5, 161.4, 168.8. HRMS (ESI+) (m/z): [M+H]⁺ calcd. for C₂₄H₂₄NO₄S, 422.1421; found, 422.1411.

(3R)-7-[(4-methoxynaphthalen-1-yl)methyl]-8-cyclopropyl-5-oxo-2,3-dihydro-5H-[1,3]thiazolo[3,2-a]pyridine-3-carboxylate (KSK85). The title compound was prepared by adaptation of a reported procedure (Chorell E, Pinkner J S, Phan G, Edvinsson S, Buelens F, Remaut H, Waksman G, Hultgren S J, Almqvist F. 2010. Design and synthesis of C-2 substituted thiazolo and dihydrothiazolo ring-fused 2-pyridones: pilicides with increased antivirulence activity. Journal of Medicinal Chemistry 53:5690-5695). Lithium hydroxide (1.0 M aqueous solution, 0.380 mL, 0.380 mmol) was added to a cooled solution (0° C.) of the methyl ester S4 (80 mg, 0.190 mmol) in THF (8 mL) and stirred at room temperature overnight. The reaction was acidified (circa pH 1) with aqueous HCl (1.0 M), extracted with EtOAc, dried (Na₂SO₄) and the solvent removed under reduced pressure. Purification by flash chromatography (SiO₂, MeOH/CH₂Cl₂/AcOH, 95:5:1 to MeOH/CH₂Cl₂, 97:3) and subsequent freeze-drying (H₂O:MeCN; ˜3:1) afforded the product as a white solid (62 mg, 80%). [α]_{D}²⁰=−5.2 (c=0.6, CHCl₃:MeOH; 9:1). ¹H NMR (400 MHz, DMSO-d₆) δ=0.60-0.69 (m, 1H), 0.72-0.79 (m, 1H), 0.85-0.98 (m, 2H), 1-69-1.77 (m, 1H), 3.50 (dd, J=1.8, 11.9 Hz, 1H), 3.79 (dd, J=9.0, 11.9 Hz, 1H), 3.98 (s, 3H), 4.30 (d, J=17.5 Hz, 1H), 4.39 (d, J=17.4 Hz, 1H), 5.23 (s, 1H), 5.32 (dd, J=1.8, 9.1 Hz, 1H), 6.97 (d, J=8.0 Hz, 1H), 7.30 (d, J=7.9 Hz, 1H), 7.50-7.57 (m, 2H), 7.77-7.80 (m, 1H), 8.20-8.24 (m, 1H), 13.36 (br s, 1H). ¹³C NMR (100 MHz, DMSO-d₆) δ=7.7, 7.9, 11.2, 31.9, 35.4, 56.0, 63.1, 104.5, 112.3, 113.8, 122.6, 124.6, 125.6, 125.7, 126.6, 127.3, 128.3, 132.8, 148.4, 154.5, 157.1, 160.4, 170.2. HRMS (ESI+) (m/z): [M+Na]⁺ calcd. for C₂₃H₂₁NNaO₄S, 430.1089; found, 430.1071.

### Example 2

### (3R)-9-cyclopropyl-5-oxo-7-phenyl-2,3,5,7-tetrahydropyrrolo[3,4-d][1,3]thiazolo[3,2-a]pyridine-3-carboxylic acid (GKP42).

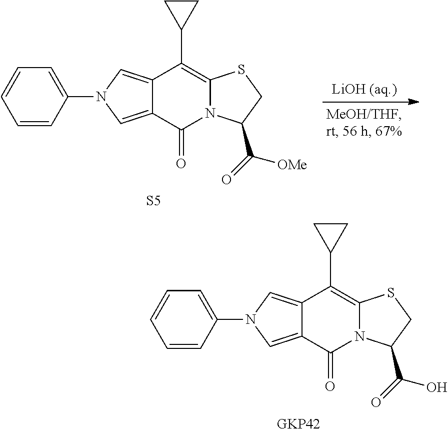 [SMILES: COC(=O)[C@@H]1CSc2c(C3CC3)c3cn(-c4ccccc4)cc3c(=O)n21 ; "S5" ; O=C(O)[C@@H]1CSc2c(C3CC3)c3cn(-c4ccccc4)cc3c(=O)n21 ; "GKP42"]

(3R)-9-cyclopropyl-5-oxo-7-phenyl-2,3,5,7-tetrahydropyrrolo[3,4-d][1,3]thiazolo[3,2-a]pyridine-3-carboxylic acid (GKP42). The title compound was prepared by adaptation of a reported procedure (Chorell E, Pinkner J S, Phan G, Edvinsson S, Buelens F, Remaut H, Waksman G, Hultgren S J, Almqvist F. 2010. Design and synthesis of C-2 substituted thiazolo and dihydrothiazolo ring-fused 2-pyridones: pilicides with increased antivirulence activity. Journal of Medicinal Chemistry 53:5690-5695). Lithium hydroxide (1.0 M aqueous solution, 0.606 mL, 0.606 mmol) was added to a cooled solution (0° C.) of the methyl ester S5 (111 mg, 0.303 mmol) in MeOH/THF (1:2; 6 mL) and stirred at room temperature for 48 h, after which additional lithium hydroxide (1.0 M aqueous solution, 0.606 mL, 0.606 mmol) was added and the reaction stirred for a further 8 h. The reaction was cooled to 0° C., acidified with aqueous HCl (0.1 M, 20 mL), extracted with 5% MeOH in CH₂Cl₂ (4×25 mL) and the solvent removed under reduced pressure. Purification by HPLC (mobile phase: MeCN/H2O with 0.75% formic acid each, 35-100% for 40 min; t_{R}=21.48 min) and subsequent freeze-drying (H₂O:MeCN; ˜3:1) gave the product as a pale orange solid (38 mg, 67%). [a]_{D}²⁰=−35.8 (c=0.6, DMSO-d₆). ¹H NMR (400 MHz, DMSO-d₆) δ=0.63-0.77 (m, 2H), 0.83-0.93 (m, 2H), 1.65-1.74 (m, 1H), 3.48 (dd, J=1.2, 11.7 Hz, 1H), 3.74 (dd, J=8.2, 11.7 Hz, 1H), 5.42 (dd, J=1.2, 8.2 Hz, 1H), 7.36-7.41 (m, 1H), 7.47 (d, J=2.3 Hz, 1H), 7.50-7.56 (m, 2H), 7.78-7.81 (m, 2H), 8.09 (d, J=2.3 Hz, 1H). ¹³C NMR (100 MHz, DMSO-d₆) δ=5.6, 5.9, 10.9, 31.4, 60.9, 104.7, 109.7, 115.7, 118.3, 120.9, 127.1, 127.2, 129.8, 134.8, 139.3, 157.9, 170.5. HRMS (ESI+) (m/z): [M+H]⁺ calcd. for C₁₉H₁₇N₂O₃S, 353.0954; found, 353.0925.

B. Biological Activity

The disclosed inhibitors can disrupt T4SS-dependent processes in multiple bacterial pathogens. Helicobacter pylori exploits a pilus associated with the cag T4SS to inject an oncogenic effector protein (CagA) and peptidoglycan into gastric epithelial cells. In H. pylori, KSK85 impedes biogenesis of the pilus appendage associated with the cag T4SS, while C10 disrupts cag T4SS activity without perturbing pilus assembly. In addition to the effects in H. pylori, the disclosed compounds disrupt inter-bacterial DNA transfer by conjugative T4SSs in Escherichia coli, and impede vir T4SS-mediated DNA delivery by Agrobacterium tumefaciens in a plant model of infection. C10 effectively disarms dissemination of a de-repressed IncF plasmid into a recipient bacterial population, thus demonstrating the potential of these compounds in mitigating the spread of antibiotic resistance determinants driven by conjugation. Thus, the disclosed compounds can impair delivery of both effector protein and DNA cargos by diverse T4SSs.

Identification of small molecules that disrupt H pylori cag type IV effector delivery. A functionally active cag T4SS in H. pylori induces secretion of the proinflammatory cytokine IL-8 and activation of NF-κB signaling when H. pylori is co-cultured with gastric epithelial cells. A series of peptidomimetic small molecules (Table S1) were screened for their capacity to inhibit secretion of IL-8 and activation of NF-κB signaling in H. pylori-gastric epithelial cell culture (FIG. 1, FIG. 5A). This focused library consisted of twenty-two compounds that contain a central peptidomimetic 2-pyridone motif (Table S1). In addition to compounds known to affect the assembly of type 1 pili or curli in E. coli (e.g., EC240 and FN075), analogues prepared when developing synthetic methodologies were included (e.g., C10, MS218 and MS383), and from natural product inspired diversity-oriented synthesis (e.g., MS542 and MS610). None of the compounds with known activity against CUP pilus assembly in E. coli affected cag T4SS activity at the tested concentrations. Two compounds significantly reduced cag T4SS-dependent IL-8 secretion and NF-κB activation (C10 and KSK85, Table 1) in a dose-dependent manner (FIG. 1B and 1D). The effects of C10 and KSK85 were additive when both compounds were present at equal concentrations in H. pylori-gastric epithelial cell co-cultures (FIG. 1B, grey line). An inactive tricyclic analogue GKP42 (FIG. 1A) was included as a non-inhibitory compound. GKP42 had minimal or no effects on IL-8 secretion at the highest concentrations tested (FIG. 1B), and did not attenuate NF-κB activation (FIG. 1D), or exhibit activity in subsequent assays. Exposure to C10, KSK85 or GKP42 did not impact viability of gastric epithelial or bacterial cells (FIG. 5B-C) at concentrations up to 150 μM.

The inhibition of cag-T4SS-mediated IL-8 secretion and NF-κB activation by C10 and KSK85 prompted measurement of CagA delivery to gastric epithelial cells. CagA is phosphorylated at conserved tyrosine residues upon T4SS-dependent translocation into the host cell. Thus, translocated CagA can be detected by immunoblot analysis probing for the presence of phosphorylated CagA. Compared to vehicle-only and GKP42-treated controls, exposure to C10 and KSK85 significantly reduced the amount of tyrosine-phosphorylated CagA detected in cultured gastric epithelial cells (FIG. 1C, FIG. 5E). Compound treatment of AGS gastric epithelial cells in the absence of bacteria did not affect the ability of the cells to signal through the canonical TNFα pathway, which leads to activation of NF-κB and subsequent synthesis and secretion of IL-8 (FIG. 1E-F). These compounds did not prevent bacterial adherence to gastric epithelial cells at concentrations ranging from 25 μM-150 μM (FIG. 5D), indicating that the observed reduction in cag T4SS activity was not a consequence of fewer bacterial cells establishing contact with gastric epithelial cells. The compounds did not impair type V secretion system-mediated release of the cytotoxin VacA into cell culture supernatants (FIG. 5F), suggesting that C10 and KSK85 did not broadly impact bacterial secretory processes. C10 and KSK85 inhibited cag T4SS-mediated NF-κB activation by H. pylori strains of different phylogeographic origins (FIG. 5G-H). These data indicate that the observed compound effects were not strain-specific. Further consideration of structure-activity relationships underlying the capactity of the compounds to inhibit cag T4SS function is given below.

The molecules employed in screening provided initial information on the structure-activity relationship underlying attenuation of cag T4SS function (Table S1). The two active compounds (C10 and KSK85), incorporated a central bicyclic ring-fused thiazolino 2-pyridone, a C-3 carboxylic acid, a C-8 cyclopropyl substituent and a C-7 CH2-(1-naphthyl) which varied through the methoxy substituent present in KSK85. Introducing a benzylic sub stituent into the C-2 position on the thiazolo ring, an important feature for pilicide activity in E. coli, was not tolerated (EC240, Table S1). Similarly, increasing the size of the C-8 substituent on the 2-pyridone ring was not tolerated (PhenylC8, FN075). Appending amino moieties onto the C-6 position abolished activity, demonstrated by MS68 and NP048, while fusing the C-6 and C-7 positions ablated activity, with both the tricyclic heterocyclic analogues MS218 and GKP42 inactive.

TABLE S1
Composition of peptidomimetic 2-pyridone focused screening library

| Compound | AGS | H. pylori | cag T4SS |
|---|---|---|---|
| (Reference) | Toxicity^{b} | Toxicity^{b} | inhibition |
| 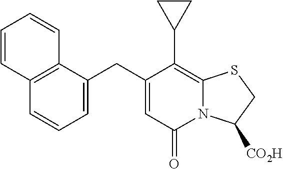 [SMILES: O=C(O)[C@@H]1CSc2c(C3CC3)c(Cc3cccc4ccccc34)cc(=O)n21] C10 | − | − | +++ |
| 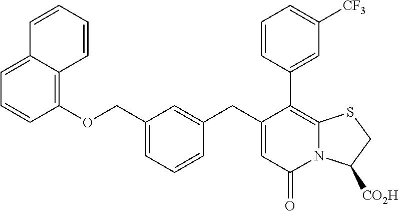 [SMILES: O=C(O)[C@@H]1CSc2c(-c3cccc(C(F)(F)F)c3)c(Cc3cccc(COc4cccc5ccccc45)c3)cc(=O)n21] CB151 | − | Toxic | − |
| 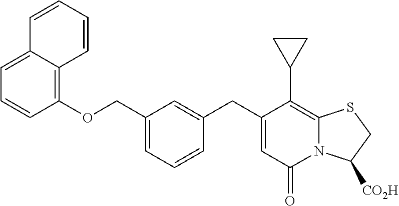 [SMILES: O=C(O)[C@@H]1CSc2c(C3CC3)c(Cc3cccc(COc4cccc5ccccc45)c3)cc(=O)n21] CB160 | − | Toxic | − |
| 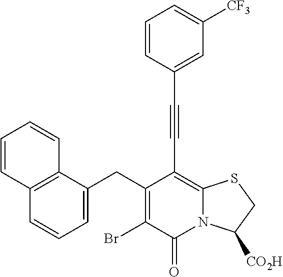 [SMILES: O=C(O)[C@@H]1CSc2c(C#Cc3cccc(C(F)(F)F)c3)c(Cc3cccc4ccccc34)c(Br)c(=O)n21] CB220 | − | − | − |
| 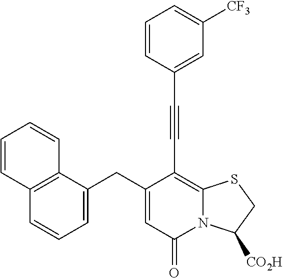 [SMILES: O=C(O)[C@@H]1CSc2c(C#Cc3cccc(C(F)(F)F)c3)c(Cc3cccc4ccccc34)cc(=O)n21] CB223 | − | − | − |
| 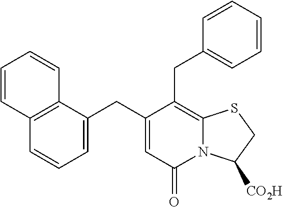 [SMILES: O=C(O)[C@@H]1CSc2c(Cc3ccccc3)c(Cc3cccc4ccccc34)cc(=O)n21] EC016 | − | − | − |
| 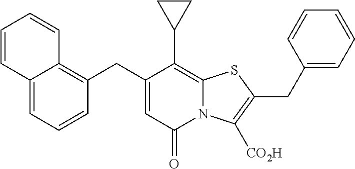 [SMILES: O=C(O)c1c(Cc2ccccc2)sc2c(C3CC3)c(Cc3cccc4ccccc34)cc(=O)n12] EC240 (1) | − | Toxic | − |
| 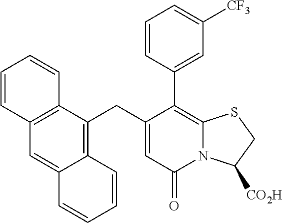 [SMILES: O=C(O)[C@@H]1CSc2c(-c3cccc(C(F)(F)F)c3)c(Cc3c4ccccc4cc4ccccc34)cc(=O)n21] EC312 | − | − | − |
| 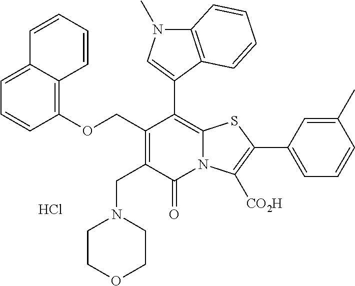 [SMILES: Cc1cccc(-c2sc3c(-c4cn(C)c5ccccc45)c(COc4cccc5ccccc45)c(CN4CCOCC4)c(=O)n3c2C(=O)O)c1 ; Cl] EC341 | − | Toxic | − |
| 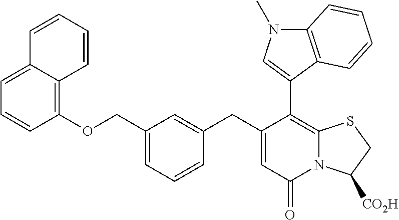 [SMILES: Cn1cc(-c2c(Cc3cccc(COc4cccc5ccccc45)c3)cc(=O)n3c2SC[C@H]3C(=O)O)c2ccccc21] EC369 | Toxic | − | − |
| 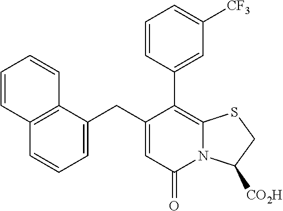 [SMILES: O=C(O)[C@@H]1CSc2c(-c3cccc(C(F)(F)F)c3)c(Cc3cccc4ccccc34)cc(=O)n21] FN075 | − | Toxic | − |
| 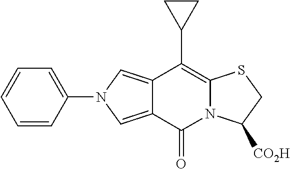 [SMILES: O=C(O)[C@@H]1CSc2c(C3CC3)c3cn(-c4ccccc4)cc3c(=O)n21] GKP42 | − | − | − |
| 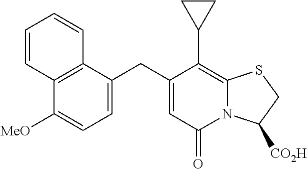 [SMILES: COc1ccc(Cc2cc(=O)n3c(c2C2CC2)SC[C@H]3C(=O)O)c2ccccc12] KSK85 This disclosure | − | − | +++ |
| 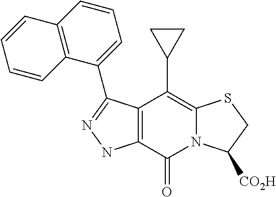 [SMILES: O=C(O)[C@@H]1CSc2c(C3CC3)c3c(-c4cccc5ccccc45)n[nH]c3c(=O)n21] MS218 | − | − | − |
| 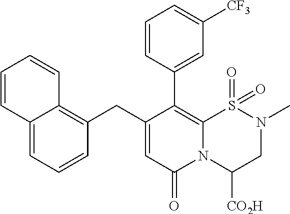 [SMILES: CN1CC(C(=O)O)n2c(c(-c3cccc(C(F)(F)F)c3)c(Cc3cccc4ccccc34)cc2=O)S1(=O)=O] MS383 | − | − | − |
| 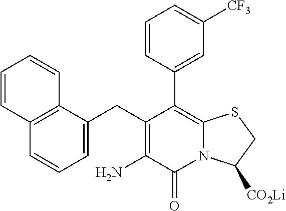 MS400 | − | − | − |
| 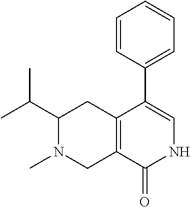 [SMILES: CC(C)C1Cc2c(-c3ccccc3)c[nH]c(=O)c2CN1C] MS542 | − | − | − |
| 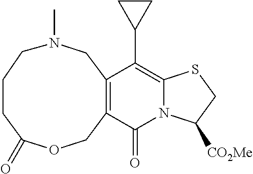 [SMILES: COC(=O)[C@@H]1CSc2c(C3CC3)c3c(c(=O)n21)COC(=O)CCCN(C)C3] MS610 (2) | − | − | − |
| 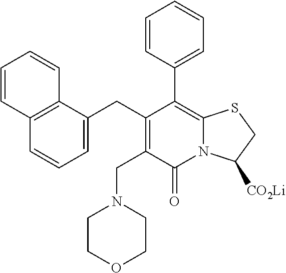 NP047 | − | − | − |
| 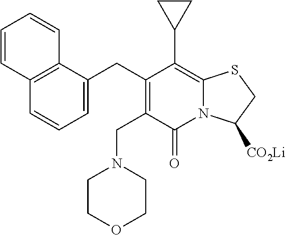 NP048 | − | − | − |
| 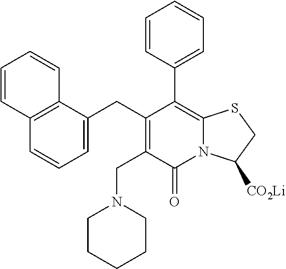 NP154 | − | − | − |
| 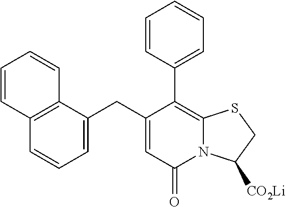 PhenylC8 | − | − | − |

^{a}Compounds (150 μM) were screened for phenotypic disruption of cag T4SS activity as measured by a significant decrease in IL-8 secretion or a significant decrease in NF-kB activation induced by WT H. pylori in co-culture with AGS gastric epithelial cells.
^{b}Compounds were determined to be toxic to either AGS cells or H. pylori by a significant decrease in cellular ATP at 18 h of incubation.

KSK85 impairs T4SS-associated pilus formation. The peptidomimetic 2-pyridone scaffold used to generate C10 and KSK85 was designed to ablate pilus biogenesis in uropathogenic E. coli. The effects of C10 and KSK85 on the assembly of cag T4SS-associated pili at the bacteria-host cell interface was investigated. H. pylori exposed to C10, GKP42, or vehicle (FIG. 2A-C) produced similar numbers of cag T4SS pili, while treatment with KSK85 abrogated pilus formation in the majority of H. pylori cells (FIG. 2D-F). A small proportion of KSK85-treated H. pylori formed pili, which could account for the low levels of CagA translocation in KSK85-treated samples (FIG. 1C, FIG. 5E); however, the proportion of piliated cells, as well as the number of pili produced by each bacterial cell, was markedly reduced compared to C10-, GKP42- and vehicle-treated samples (FIG. 2E-F).

CagA attenuates the disruptive effects of C10 and KSK85 on T4SS activity. Activation of NF-κB signaling and induction of IL-8 secretion can occur in response to translocated CagA and/or peptidoglycan. It is thus possible that C10 and KSK85 also influence the translocation of peptidoglycan, or other unidentified effector molecules. To assess whether the inhibitory effects of the compounds on NF-κB signaling and IL-8 secretion were attributable to blocking one or both of types of translocation, the effects of KSK85 and C10 on an H. pylori ΔcagA mutant were evaluated. The effects of C10 on H. pylori ΔcagA were the same as those observed in the WT strain (FIG. 3A). These results suggest that the inhibitory effects of C10 on NF-κB activation are primarily attributable to blocking translocation of peptidoglycan or additional effector molecules (FIG. 3A). The ability of KSK85 to disrupt cag T4SS-mediated NF-κB activation was reduced compared to the inhibitory effects of C10 (FIG. 3A vs. FIG. 3B); however, the inhibitory effects of KSK85 were significantly enhanced in the cagA mutant strain (p=0.004, paired t-test) compared to the WT strain (FIG. 3B). To investigate the basis for this phenomenon, CagA's importance for pilus assembly was analyzed, and it was observed that the ΔcagA mutant produced T4SS-associated pili at levels similar to WT (FIG. 6A). Thus, although KSK85 inhibits formation of cag T4SS pili, loss of CagA does not preclude elaboration of pilus structures that are targeted by KSK85. In addition, the absence of CagA did not influence adherence of bacteria to the gastric epithelial surfaces (FIG. 6B), indicating that pilus-associated CagA does not facilitate binding of H. pylori to target host cells. Collectively, these data suggested that the inhibitory activity of KSK85 is reduced in the presence of CagA, potentially through CagA-compound interactions.

H. pylori exploits the surface of gastric epithelial cells as a replicative niche by using cag T4SS-dependent processes to control cell polarity. The effects of the compounds on replication of H. pylori co-cultured with gastric epithelial cells was evaluated. Consistently fewer CFUs of a ΔcagE mutant (deficient in an essential ATPase of the cag T4SS, and defective in pilus production) were recovered from AGS cell co-culture at 6 hours post-infection, compared to recovery of the WT strain, regardless of compound exposure (FIG. 3C). The ΔcagE mutant exhibited WT adherence properties (FIG. 6B), ruling out the possibility that the differential recovery of bacteria was attributable to differences in adherence. Similar numbers of CFUs of the WT strain and cagA mutant were recovered from control co-cultures (treated with DMSO) (FIG. 3C). Studies revealed that the number of ΔcagA CFUs recovered from co-culture at 6 hours post-infection were reduced at least 10-fold in the compound-treated wells, compared to the control-treated bacteria (FIG. 3C). This result indicated that loss of CagA enhances the T4SS inhibitory capacity of the compounds when H. pylori are in contact with host cells. However, it is possible that the reduction in recovered ΔcagA CFUs does not stem from altered host cell phenotypes that arise as a consequence of CagA translocation, which may confer survival benefits to the bacterium at this host-microbe interface.

C10 and KSK85 significantly reduce T4SS-dependent DNA transfer in divergent proteobacteria. Given the incredible diversity of T4SS across phyla with respect to both function and apparatus architecture, C10 and KSK85 were evaluated for attenuation of T4SS processes in other bacterial species. The ability of C10 and KSK85 to prevent DNA transfer through the IncN group conjugative T4SS encoded by pKM101 and the IncF group R1-16 conjugative T4SS in E. coli was analyzed. Compared to vehicle controls, C10 and KSK85 reduced the DNA conjugation efficiency of pKM101 in a statistically significant manner (FIG. 4A). Compound treatment did not impair E. coli growth (FIG. 7A), suggesting that the observed decrease in numbers of trans-conjugants was not due to reduced replication of the bacteria in the presence of compounds. Analysis of DNA conjugation efficiency of the de-repressed R1-16 system (lacking a regulatory element that restricts DNA conjugation) demonstrated that C10 reduced DNA transfer, whereas KSK85 did not (FIG. 4A), suggesting that R1-16 de-repression can partially compensate for the T4SS inhibitory effects of KSK85.

The ability of C10 and KSK85 to ablate Agrobacterium vir T4SS-mediated DNA translocation into recipient plant cells was analyzed using an intron-containing β-glucuronidase (GUS) gene reporter assay. Species of the genus Agrobacterium encode type IV conjugation systems that facilitate the delivery of plasmid-derived transfer DNA (T-DNA) into target plant cells; the T-DNA is subsequently integrated randomly into the plant cell genome. Conjugation-mediated transfer of T-DNA from A. tumefaciens to plants can be readily monitored using an Agroinfiltration system developed for tobacco. This method relies on the infiltration of A. tumefaciens into the interstitial spaces of a leaf, followed by incubation to allow for T-DNA transfer, integration of T-DNA into the nuclear DNA of transformed plant cells, and T-DNA expression. In the assay, use of the intron-GUS expression cassette permits GUS enzyme production only by suitably transformed plant cells because A. tumefaciens lacks the splicing machinery necessary for removal of introns. Qualitative analysis of GUS activity can be performed by staining using X-Gluc (5-bromo-4-chloro-3-indolyl-beta-D-glucuronic acid, cyclohexylammonium salt) as substrate (FIG. 8A). Using a fluorescence-based assay that measures GUS enzymatic activity, it was determined that T-DNA transfer and genomic integration could be reliably measured and quantified at 48 hours post-infection.

Bacterial Strains and Growth Conditions. H. pylori strains 26695, G27, 98-10, and isogenic 26695 mutants were grown on trypticase soy agar supplemented with 5% sheep blood (BD Biosciences) or Brucella broth supplemented with 5% fetal bovine serum at 37° C. in 5% atmosphere CO₂. E. coli MG1655 harboring the conjugative plasmid pKM101, E. coli MS411 harboring the R1-16 conjugative plasmid, and E. coli WM1652 recipient cells were grown on LB agar or broth supplemented with ampicillin (50 μg/ml), kanamycin (50 μg/ml), or tetracycline (20 μg/ml), respectively. Agrobacterium tumefaciens C58 and its derivative GV3101 were maintained on LB plates supplemented with rifampicin (25 μg/ml) at 28° C. A. tumefaciens GV3101 harboring pCAMBIA vectors were maintained on LB containing rifampicin (25 μg/ml) and kanamycin (100 μg/ml).

Cell culture. AGS human gastric epithelial cells and the AGS NF-κB luciferase-reporter cell line were grown in 5% CO₂ in RPMI medium supplemented with 10% FBS, 2 mM L-glutamine, and 10 mM HEPES buffer (complete RPMI).

Cell viability assays. H. pylori, A. tumefaciens, E. coli, or human cell lines were grown in complete RPMI or Brucella broth supplemented with the indicated compounds or DMSO for 6-24 h. Cell viability was assessed using CellTiter-Glo (Promega) according to the manufacturer's protocol.

IL-8 induction assays. Quantitation of IL-8 secretion by gastric epithelial cells in contact with H. pylori was performed as previously described (Shaffer C L, Gaddy J A, Loh J T, Johnson E M, Hill S, Hennig E E, McClain M S, McDonald W H, Cover T L. 2011. Helicobacter pylori exploits a unique repertoire of type IV secretion system components for pilus assembly at the bacteria-host cell interface. PLoS Pathog 7:e1002237) and as follows. H. pylori was harvested from blood agar plates after 48 h of growth, re-suspended in complete RPMI at approximately 10⁹ CFUs/ml and incubated for 1 h with shaking in the presence of the indicated compounds or DMSO at a concentration of 150 μM. The RPMI medium was removed from AGS cell monolayers and was replaced with an equivalent volume of complete RPMI supplemented with compound (ranging from 150 μM to 25 μM), or equivalent volume of DMSO vehicle alone immediately prior to inoculation with H. pylori. AGS cell monolayers (80-90% confluent) were infected at a multiplicity of infection (MOI) of 100:1. Triplicate wells per condition were inoculated. Following 4 h incubation with H. pylori, AGS cell supernatants were collected, and IL-8 levels were quantified by anti-human IL-8 ELISA (R&D). Levels of IL-8 secreted by AGS cells in response to H. pylori infection in the presence of individual compounds were compared to the levels of IL-8 secreted in the DMSO control wells (set to 100% in all experiments).

Quantitation of NF-κB signaling activation. AGS cells stably transfected with an NF-κB-luciferase reporter (AGS NF-κB-luc) were cultured for 24 h and treated as described for the IL-8 induction studies. After 4 h co-culture with H. pylori, cell monolayers were washed with sterile PBS, and cell monolayer extracts were obtained by passive cell lysis. NF-κB-luciferase activity was measured by the Steady-Glo Luciferase Assay System (Promega) on a BioTek Synergy 4 plate reader, and plates were normalized to the DMSO vehicle control for each dilution series. AGS NF-κB-luc cells exposed to compounds in the absence of H. pylori were stimulated for 1 h with 10 ng/ml TNFα and assayed by the Steady-Glo system to validate that compounds do not impact NF-κB signaling.

CagA translocation assay. Translocation of CagA into AGS cells was analyzed as described in (Shaffer C L, Gaddy J A, Loh J T, Johnson E M, Hill S, Hennig E E, McClain M S, McDonald W H, Cover T L. 2011. Helicobacter pylori exploits a unique repertoire of type IV secretion system components for pilus assembly at the bacteria-host cell interface. PLoS Pathog 7:e1002237) and as follows. Translocation of CagA into AGS cells was analyzed as described in (Shaffer C L, Gaddy J A, Loh J T, Johnson E M, Hill S, Hennig E E, McClain M S, McDonald W H, Cover T L. 2011. Helicobacter pylori exploits a unique repertoire of type IV secretion system components for pilus assembly at the bacteria-host cell interface. PLoS Pathog 7:e1002237). Briefly, H. pylori was pre-treated with compounds and co-cultured with AGS cells at an MOI of 100 in triplicate in complete RPMI supplemented with 150 μM compound or DMSO for 4.5 h at 37° C. in 5% CO₂ atmosphere. AGS monolayers were washed in PBS containing 2 mM sodium orthovanadate to remove non-adherent bacteria, and AGS cells were lysed in 1% NP-40 buffer containing Complete Mini (EDTA-free) Protease Inhibitor (Roche) and PhosSTOP pan phosphatase inhibitor (Roche). CagA translocation was assessed by separation of the soluble cellular fraction by SDS-PAGE and immunoblot analysis using anti-phosphotyrosine antibody (α-PY99, Santa Cruz). Levels of phospho-CagA were normalized to levels of total CagA (α-CagA, Santa Cruz) in 5 independent experiments. Densitometry analysis was performed in ImageJ.

Scanning electron microscopy. Overnight cultures of H. pylori were diluted to OD_{600˜0.3} and incubated with shaking at 37° C., 5% CO₂ for 1 h in the presence of vehicle or 150 μM compound and co-cultured with AGS cells on tissue culture-treated coverslips (BD Biosciences) at an MOI of 100 in the presence of 150 μM compound or vehicle. H. pylori-AGS cell co-cultures were processed and imaged.

Bacterial adherence to gastric epithelial cells. Compound pre-treated H. pylori were added to AGS cells in the presence of 150 μM compound and treated as described for IL-8 induction studies. After 4 hours of co-culture, RPMI was aspirated and the wells were washed 5 times with PBS. Adherent bacteria and cell monolayers were fixed in 4% paraformaldehyde at room temperature for 15 min, followed by blocking in 1% BSA in PBS containing 0.1% Triton X-100 for 1 h at room temperature. Bacteria were stained with antisera raised against soluble H. pylori proteins (α-H. pylori) at a dilution of 1:1000 in 1% BSA for 1 h at room temperature, followed by 5 washes in PBS and incubation in goat anti-rabbit secondary antibody conjugated to Alexa-488 (1:5000) (Life Technologies). Wells were washed 3 times in PBS and fluorescence was measured using a BioTek Synergy 4 plate reader. As a control, AGS cell monolayers were stained with primary and secondary antibody or secondary antibody alone. Average fluorescence of triplicate wells was normalized to control wells, and H. pylori adherence to AGS cells in the presence of 150 μM compound was compared to H. pylori adherence in the presence of DMSO vehicle alone.

VacA secretion assay. WT H. pylori was cultured overnight in brucella broth containing 5% FBS supplemented with either 100 μM DMSO, C10, KSK85, or GKP42 as previously described (Gonzalez-Rivera C, Algood H M, Radin J N, McClain M S, Cover T L. 2012. The intermediate region of Helicobacter pylori VacA is a determinant of toxin potency in a Jurkat T cell assay. Infect Immun 80:2578-2588). Bacterial cultures were centrifuged (2,000×g) to separate bacterial cells and cell culture supernatants. Bacterial pellets were re-suspended in PBS at a final volume equivalent to the volume of the supernatant. Equal volumes of each re-suspended cell pellet and its corresponding cell culture supernatant were added to 2×SDS buffer (Bio-Rad), and equal volumes of each sample were resolved by SDS-PAGE (Bio-Rad). Immunoblotting to probe for secreted (supernatant) and bacterial cell-associated VacA (pellet) was performed as previously described (Gonzalez-Rivera).

Field emission gun scanning electron microscopy. Samples were fixed with 2.0% paraformaldehyde, 2.5% glutaraldehyde in 0.05 M sodium cacodylate buffer, and secondarily fixed with 0.1% osmium tetroxide. Sequential dehydration was performed by washing with increasing concentrations of ethanol. Samples were dried at the critical point with a Tousimis critical point dryer. Coverslips were mounted onto aluminum stubs and sputter coated with gold-palladium and imaged with an FEI Quanta 250 FEG-SEM.

H. pylon viability in AGS cell co-culture. Compound pre-treated H. pylori were added to AGS cells at an MOI of 100 in the presence of 150 μM compound and treated as described for IL-8 induction studies. After 6 hours of co-culture, RPMI was aspirated and the wells were washed 5 times with sterile PBS to remove non-adherent bacteria. AGS cells with adherent H. pylori were dislodged in sterile PBS, and serial dilutions were plated to determine the number of H. pylori CFUs. H. pylori survival experiments were performed at least 3 times with 4 replicate wells per experimental condition.

Analysis of DNA conjugation efficiency. Overnight cultures of donor E. coli MG1655 harboring pKM101, donor E. coli MS411 harboring R1-16, and recipient E. coli MG1655 (WM1652) were grown at 37° C. with shaking at 225 rpm in LB/antibiotic, were diluted in antibiotic-free LB containing DMSO or 150 μM compound, and incubated with shaking at 37° C. to reach an OD₆₀₀ of ˜0.3. Donor and recipient cells were mixed at a ratio of 1:1 on 0.22 μM pore size mixed cellulose ester filters (Millipore) on antibiotic-free LB plates for 2 h at 37° C. Filters were aseptically removed from LB plates and incubated in 100 μl antibiotic-free LB to dislodge E. coli from membranes. Serial dilutions were plated on the appropriate antibiotic (or dual antibiotic) LB plates to determine the number of donor, recipient, and transconjugant cells. Colonies were enumerated after 24 h, and conjugation efficiencies were calculated by dividing the average number of transconjugates by the average number of donors (either pKM101 or R1-16 donor cells). Conjugation efficiency in the presence of compound is expressed as a percentage of conjugation efficiency in DMSO. Data represent 6 individual experiments for compounds C10 and KSK85, and 3 individual experiments for GKP42.

Agroinfiltration and GUS assay. Agroinfilitration and GUS fluorometric assay were performed as previously described (Jefferson R A, Kavanagh T A, Bevan M W. 1987. GUS fusions: beta-glucuronidase as a sensitive and versatile gene fusion marker in higher plants. Embo J 6:3901-3907; Wu S, Schoenbeck M A, Greenhagen B T, Takahashi S, Lee S, Coates R M, Chappell J. 2005. Surrogate splicing for functional analysis of sesquiterpene synthase genes. Plant Physiol 138:1322-1333), and as follows. To determine the efficacy of test compounds to prevent transfer of T-DNA from a Ti plasmid harbored by Agrobacterium tumefaciens to the nuclear DNA of a plant host cell, A. tumefaciens (GV3101) with and without a suitable expression vector (pCAMBIA 1305.2) was utilized. The pCAMBIA 1305.2 expression vector contains an intron-containing β-glucuronidase (GUS) gene inserted downstream of the CaMV 35S promoter (a strong, constitutive gene expression promoter in plants) into the T-DNA region of the plasmid (www.cambia.org). The pCAMBIA 1305.2 vector was transformed into A. tumefaciens GV3101 via freeze-thaw method, and recombinant GV3101 lines were selected for kanamycin resistance. A. tumefaciens with the pCAMBIA 1305.2 expression vector were subsequently grown in LB liquid growth media containing 100 μg/ml of kanamycin and 10 μg/ml of rifampicin, while control A. tumefaciens not harboring the plasmid vector were grown in LB media containing only 10 μg/ml of rifampicin. A. tumefaciens cultures were grown overnight at 28° C. to an OD₆₀₀ of 0.8. The cells were then pelleted by centrifugation (2000×g for 10 min), washed twice with water to remove residual LB, and re-suspended in infiltration buffer (10 mM MES pH 5.7, 10 mM MgCl₂) at an OD₆₀₀ of 0.8. Aliquots of the cell cultures were then incubated with test compounds at 50, 150, or 450 μM concentrations for 1 hour prior to leaf infiltration. As a vehicle control, A. tumefaciens cultures harboring the pCAMBIA expression vector were incubated in equivalent concentrations of DMSO, and DNA transfer-deficient negative controls of A. tumefaciens not harboring the Ti-expression vector were treated identically.

Agroinfiltration of homogenous bacterial cultures containing the indicated final concentration of ring-fused 2-pyridone compound was performed on 2-month old Nicotiana benthamiana plants using expanding leaves of approximately 6 cm long by 5 cm wide. Six zones (two zones for controls and four zones for each experimental condition) were infiltrated per leaf with approximately 100 μl of compound-treated A. tumefaciens suspensions infiltrated per zone. Agroinfiltration was performed using a 1 ml needleless syringe to gently inject bacterial suspensions into the plant interstitial space from the abaxial surface. The infiltration zones were visible as water soaked areas that were subsequently demarcated on upper side of each leaf. Infiltration buffer within each inoculation zone was absorbed within hours following injection; therefore, the infiltration zones were injected twice more at 12 and 24 h post-initial infiltration with infiltration buffer containing the respective test compounds at the appropriate concentrations.

Leaves containing Agroinfiltration zones were harvested after 48 h of incubation, the empirically derived time point at which T-DNA transfer had occurred for sufficient detection of β-glucuronidase (GUS) enzyme activity. GUS activity was initially assessed using X-gluc (5-bromo-4-chloro-3-indolyl-β-D-glucuronide cyclohexylammonium salt) (Goldbiotech) as a substrate in GUS staining buffer (100 mM sodium phosphate pH 7.0, 10 mM EDTA, 0.5 mM potassium ferro-cyanide, 0.5 mM potassium ferricyanide, 0.05% Triton-X-100, 2 mM X-gluc). The quantitative GUS fluorometric assay was used to measure GUS gene expression. Briefly, leaf discs were homogenized in sodium phosphate buffer, and homogenates were separated by centrifugation at 10,000×g. Total protein concentration of each cleared homogenate supernatant was determined by Bradford reagent (Bio-Rad) using bovine serum albumin (BSA) to generate a standard curve. Normalized supernatants were assayed for β-glucuronidase activity by measuring the GUS cleavage-dependent conversion of methylumbelliferyl-β-D-glucuronide (MUG) substrate to fluorescent 4-methylumbelliferone (MU). GUS activity was expressed as nanomoles of MU produced per minute per milligram of protein. Average GUS activity from at least 3 independent biological replicate plants (minimum of three leaves per plant) is expressed as a percent (%) of the DMSO vehicle control (values set to 100%)±SEM; statistical significance for each compound treated condition was established by one-way ANOVA.

Carrot disk tumor formation assay. Whole carrots were sterilized by soaking in 20% bleach for 30 min, and were subsequently sliced into disks of approximately 8-10 mm. The apical surface of each disk was placed on water agar (1.5%) medium containing no additional nutritional supplementation. Carrot disks were incubated with 100 μl of either DMSO, C10, KSK85, or GKP42 (final concentration of all compounds at 150μM) in sterile PBS for 1 h to allow for the uptake of compounds into the carrot tissue. Replicate carrot disks were inoculated with 20 μl Agrobacterium tumefaciens C58 harvested from logarithmic-phase growth (OD₆₀₀˜0.8) that had been pre-treated for 1 h with shaking at 28° C. in LB containing DMSO, C10, KSK85, or GKP42 (compounds at150 μM final concentration). Plates were sealed with parafilm and stored in the dark at room temperature for three weeks. Carrot tumors were photographed at 21 days post-inoculation. Images are representative of at least 5 individual carrot disks.

Statistical Analysis. Statistical analyses were performed in GraphPad Prism 6. Data are expressed as the mean±standard error. Data comparisons from more than two groups were analyzed by ANOVA followed by Dunnett's post-hoc test for multiple comparisons against a single control (vehicle treated samples).

TABLE 1
EC₅₀ values for the attenuation of cag T4SS-dependent
IL-8 secretion by peptidomimetic small molecules^{a}

| | C10 | KSK85 | C10/KSK85^{b} | GKP42 |
|---|---|---|---|---|
| LogEC₅₀ | −1.19 | −1.14 | −1.45 | −0.648 |
| HillSlope | −1.49 | −1.23 | −0.98 | −3.12 |
| EC₅₀ (μM) | 64.5 | 72.2 | 35.8 | 225 |

Standard Error

| | | | | |
|---|---|---|---|---|
| LogEC₅₀ | 0.0322 | 0.0337 | 0.0694 | 0.229 |
| HillSlope | 0.224 | 0.188 | 0.201 | 3.21 |

95% CI

| | | | | |
|---|---|---|---|---|
| LogEC₅₀ | −1.26 to −1.12 | −1.21 to −1.07 | −1.59 to −1.30 | −1.12 to −0.172 |
| HillSlope | −1.95 to −1.02 | −1.62 to −0.839 | −1.40 to −0.562 | −9.80 to 3.56 |
| EC₅₀ (μM) | 55.3 to 75.3 | 61.4 to 84.8 | 25.7 to 50 | 75.2 to 673 |

^{a}Compounds were added to H. pylori-gastric epithelial cell monolayers for the duration of the co-culture experiment (4.5 h), and IL-8 secretion was evaluated by anti-human IL-8 ELISA as described in the Methods. EC₅₀ values were calculated from normalized IL-8 secretion levels, where IL-8 levels obtained from compound-treated samples were expressed as a percentage of values obtained in the vehicle control wells (0.3% DMSO, final concentration for all samples). Percentages from separate biological replicate samples were normalized to a range of 10-100%, where maximal T4SS inhibition is defined as 10%, and 100% represents full T4SS activity. EC₅₀ values were calculated in GraphPad using non-linear regression of normalized IL-8 secretion values (variable slope four parameter, log(agonist) vs. response).
^{b}C10 and KSK85 were added at equal concentrations ranging from 25 μM to 150 μM each.

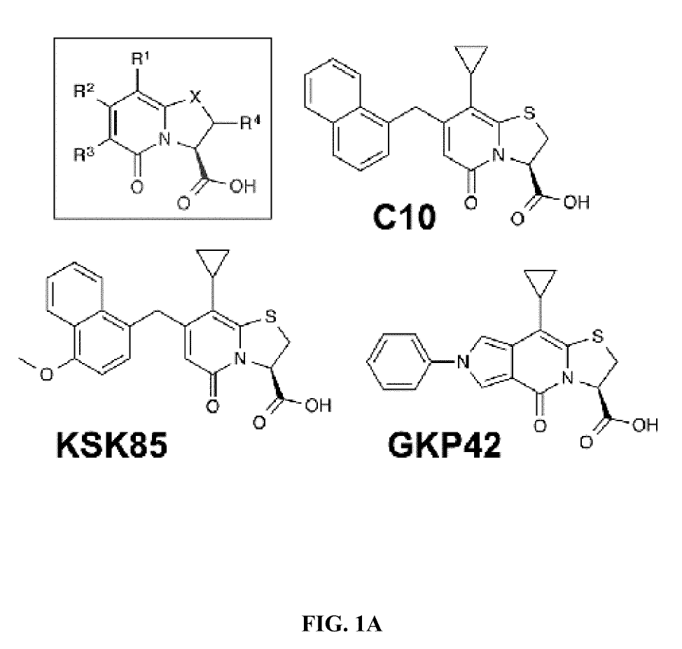

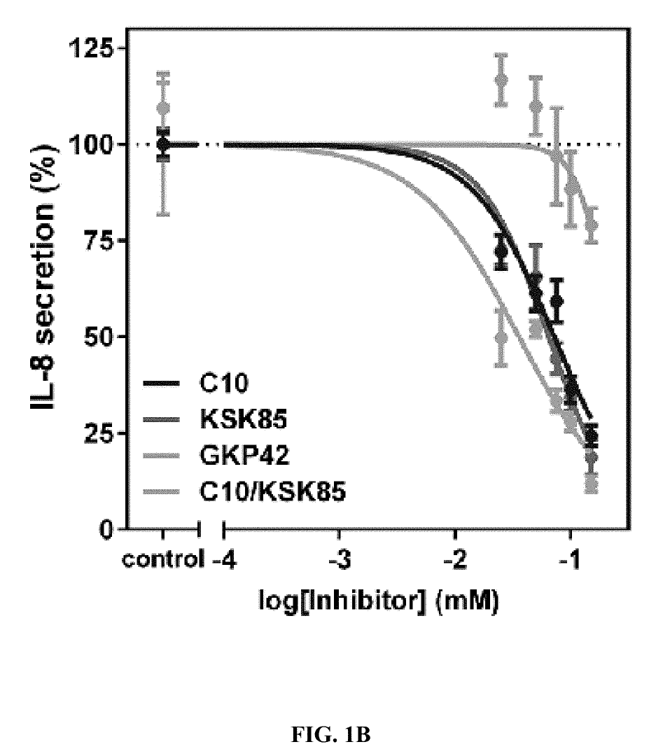

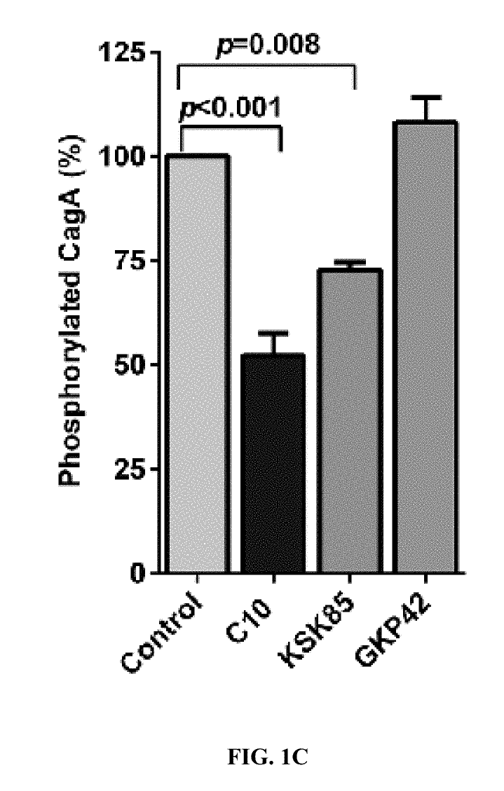

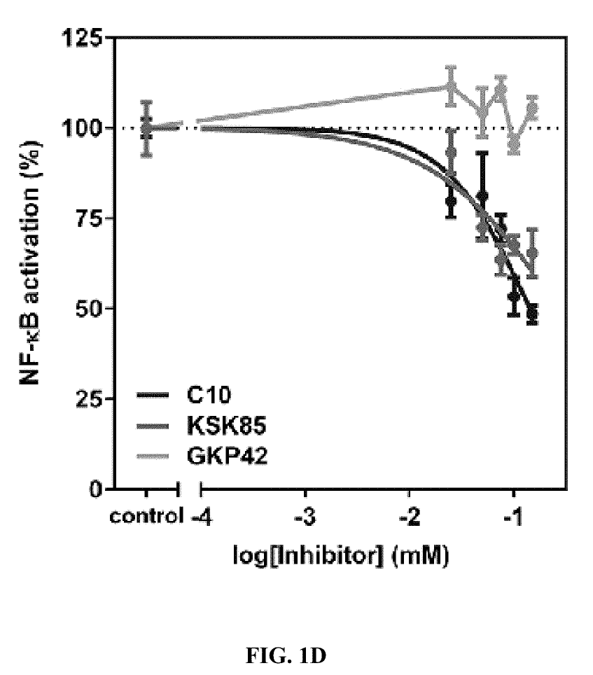

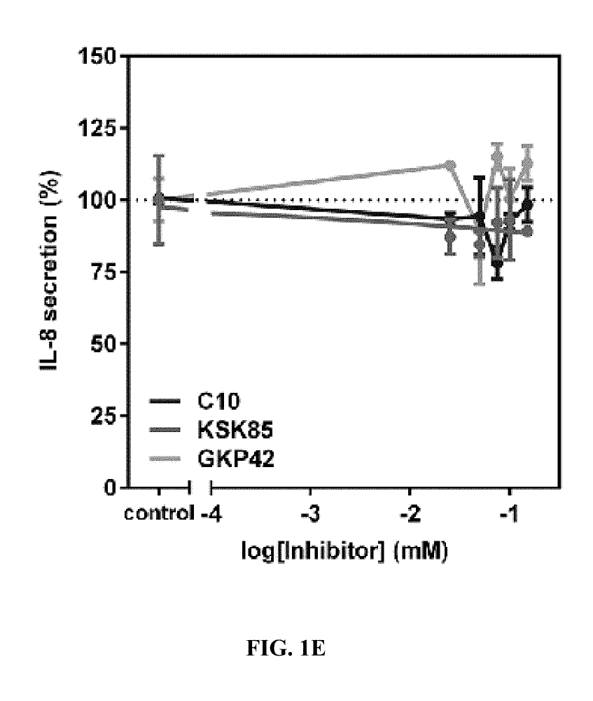

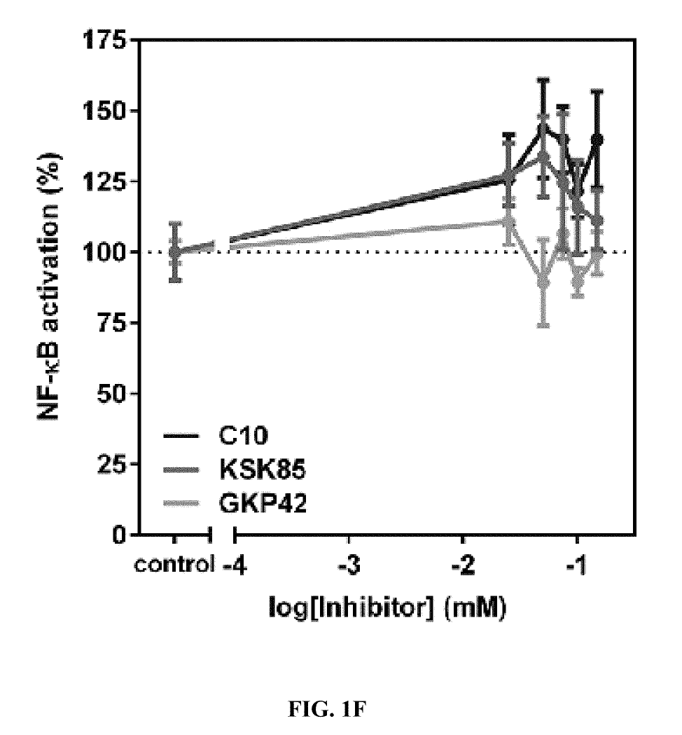

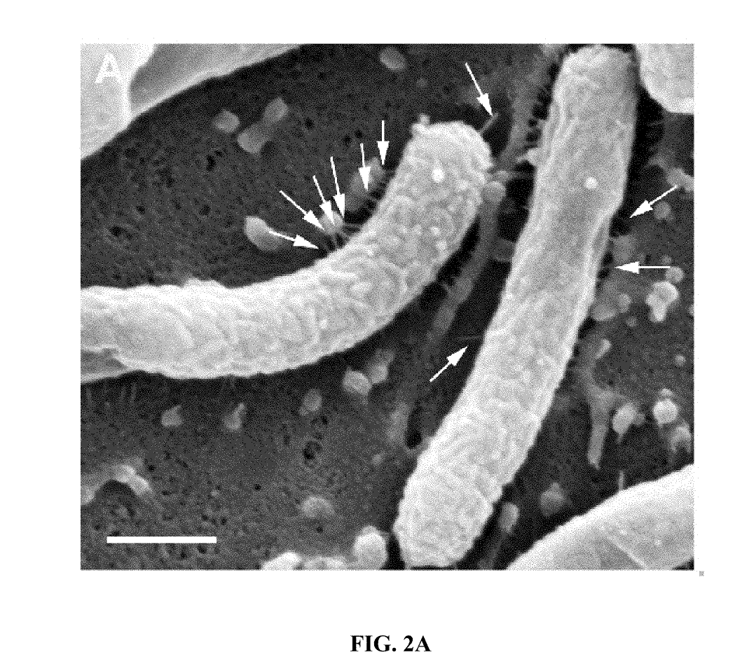

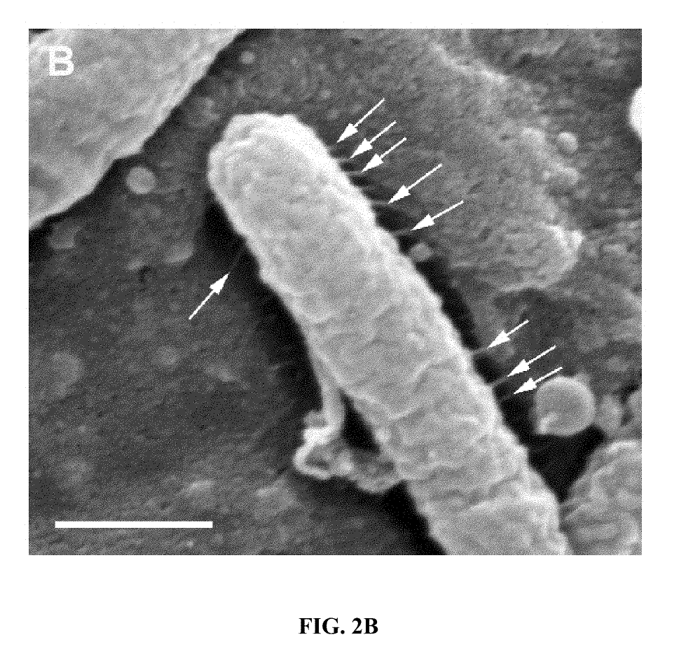

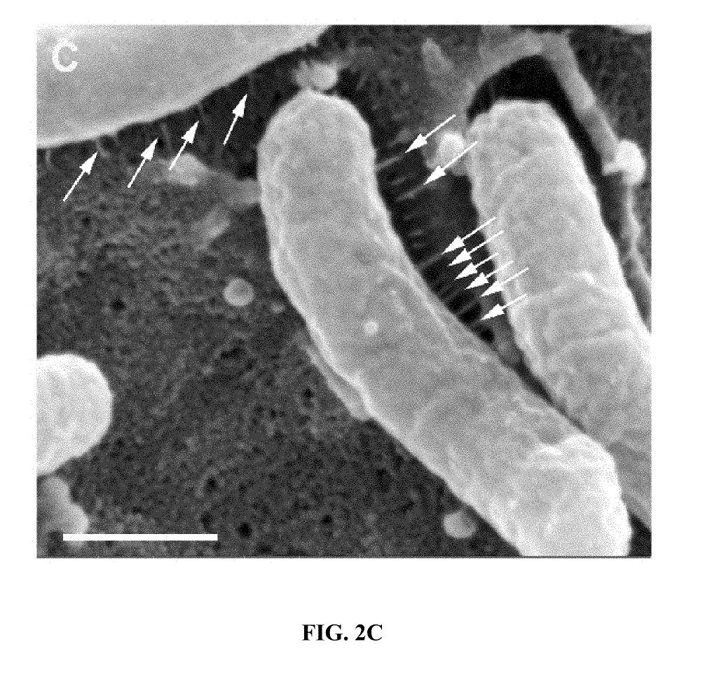

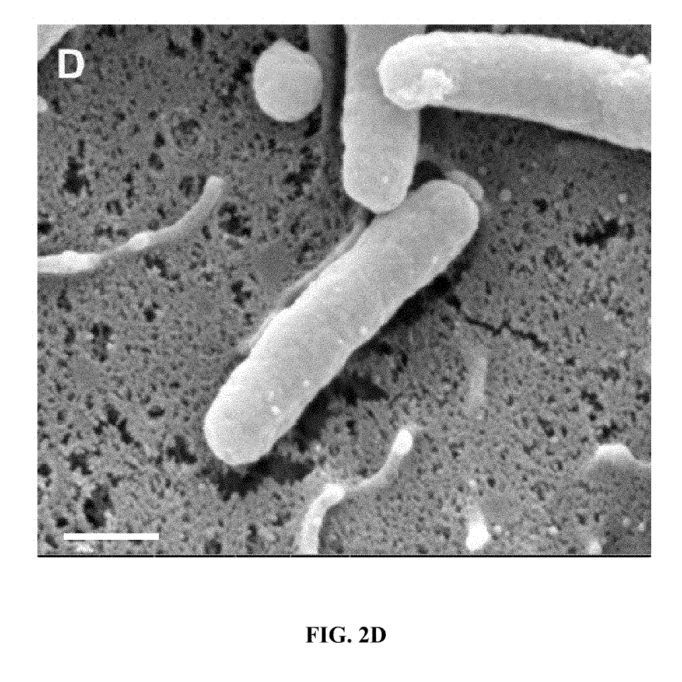

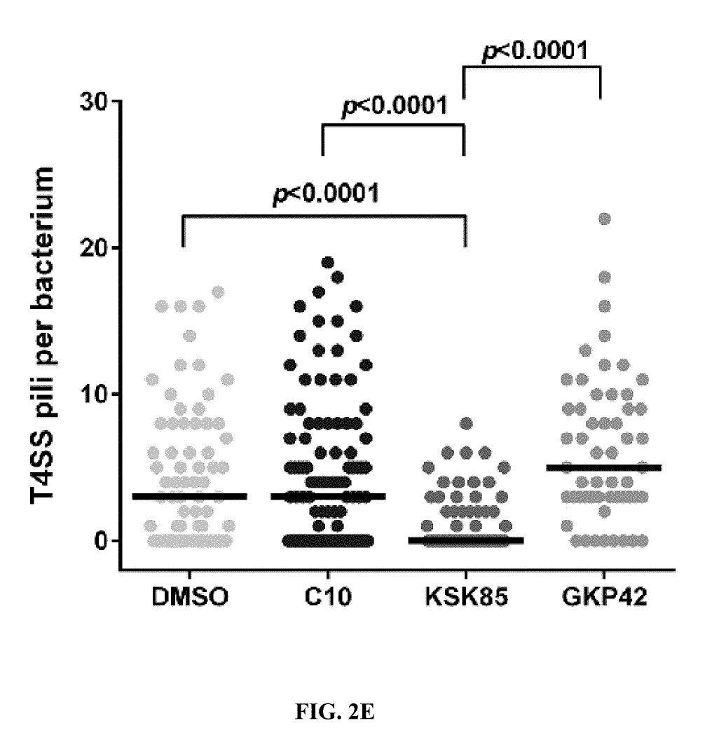

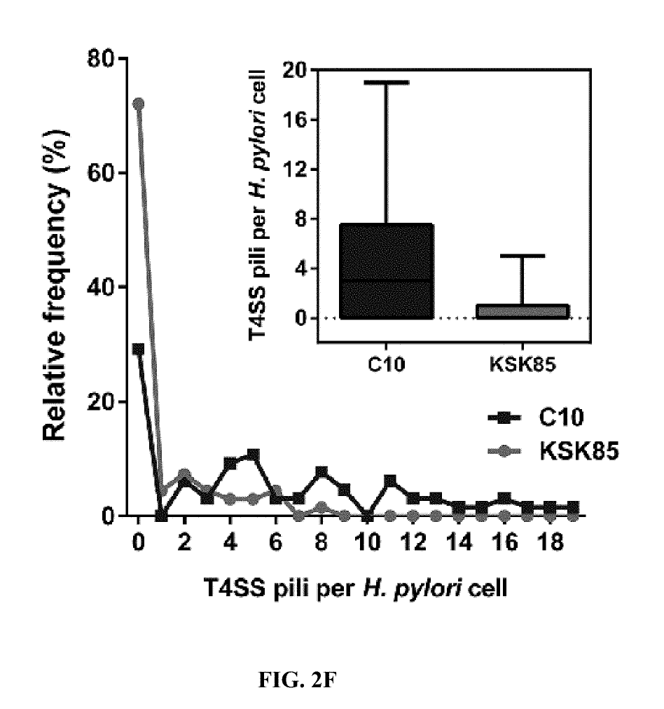

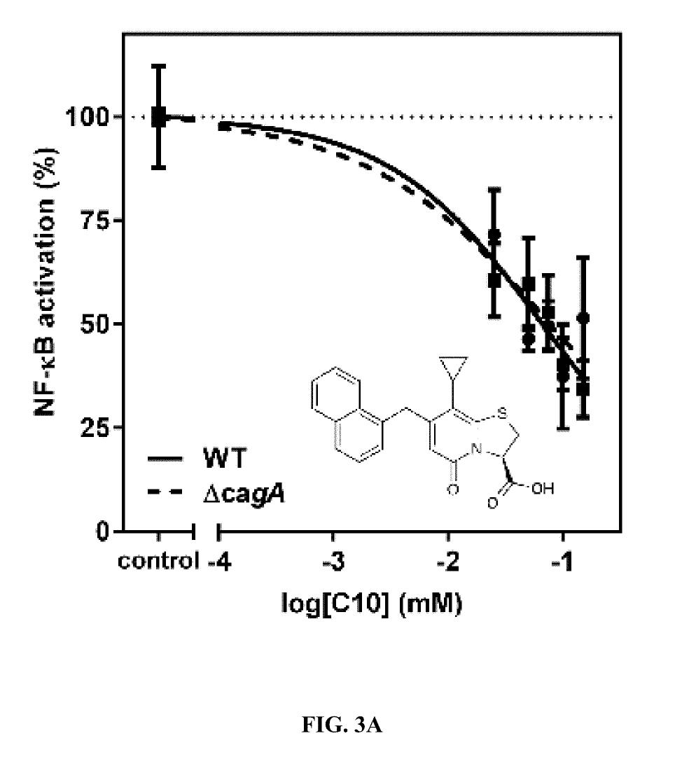

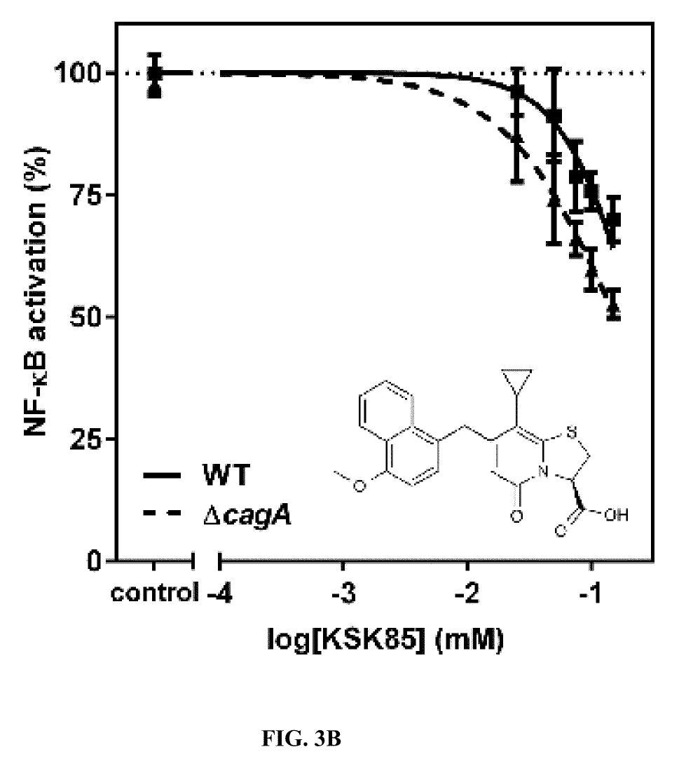

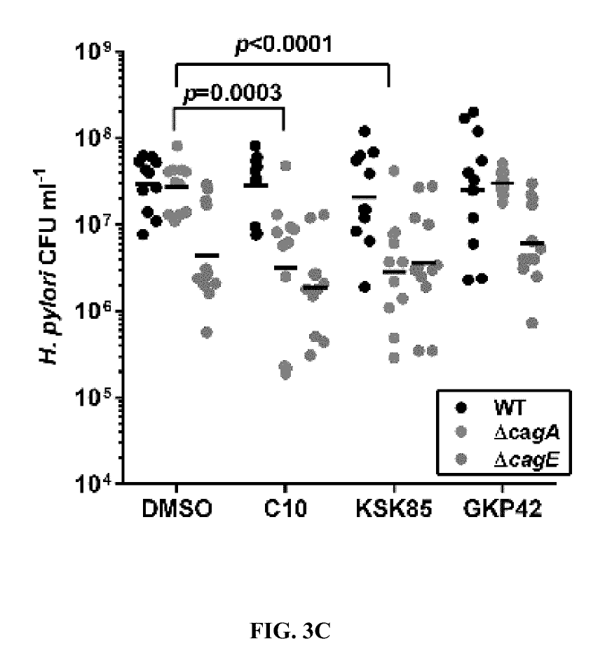

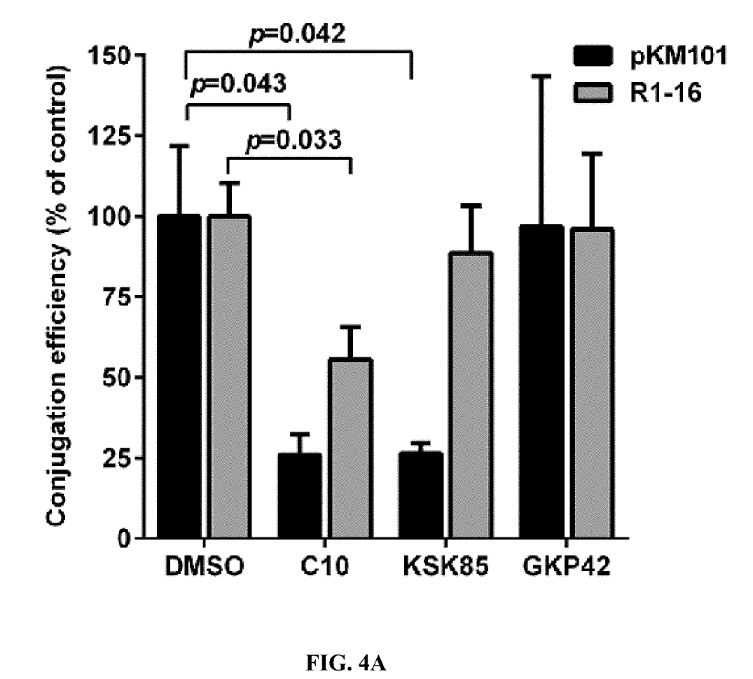

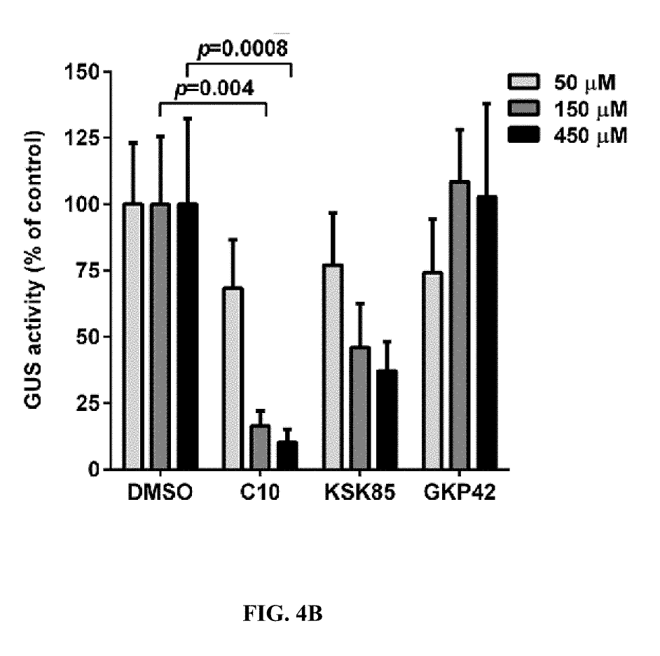

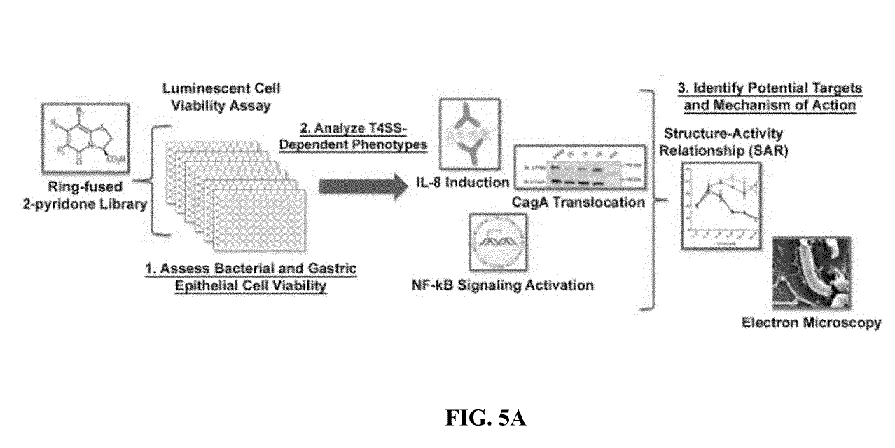

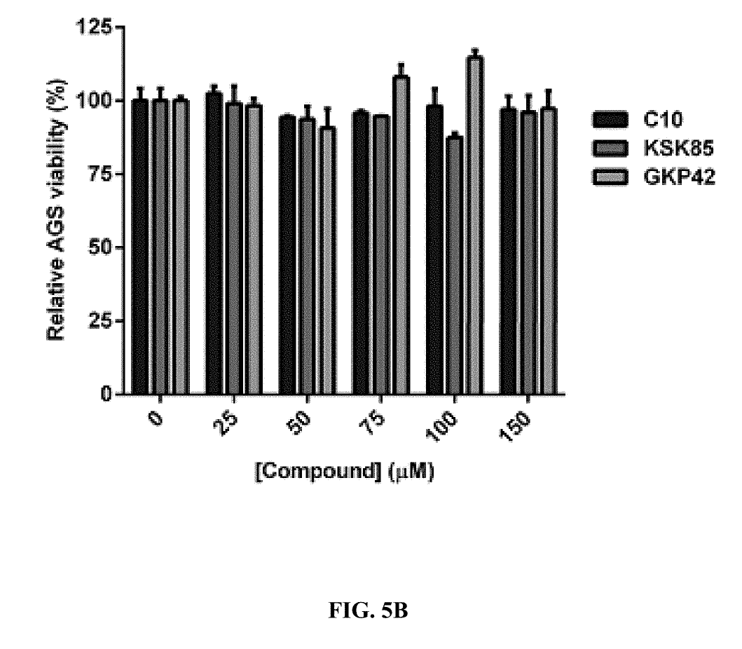

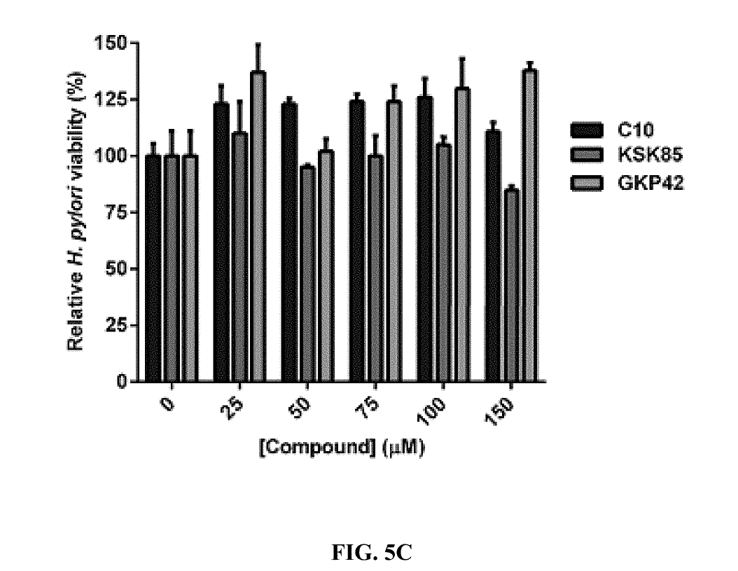

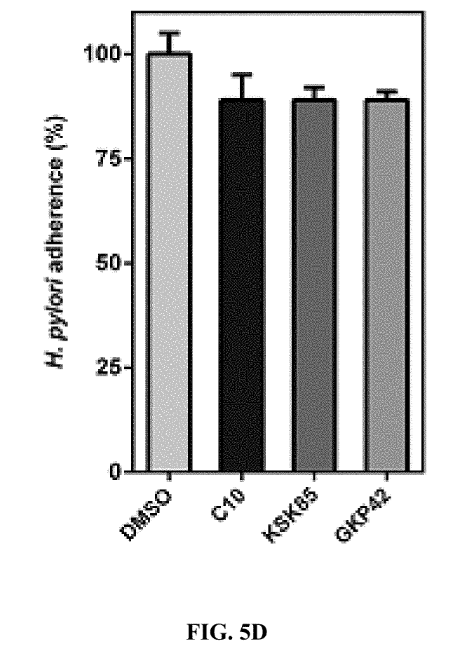

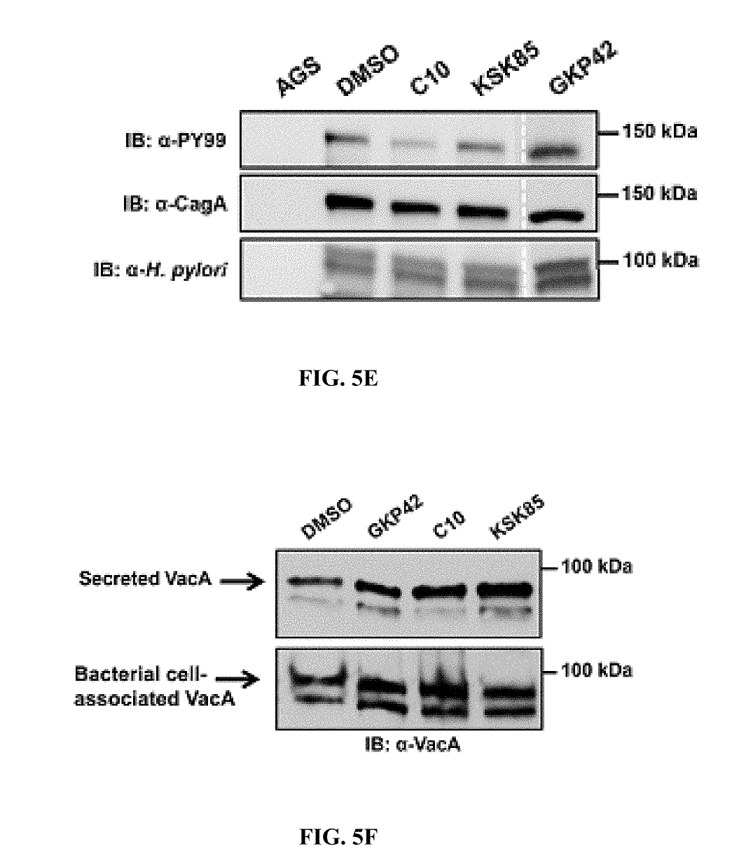

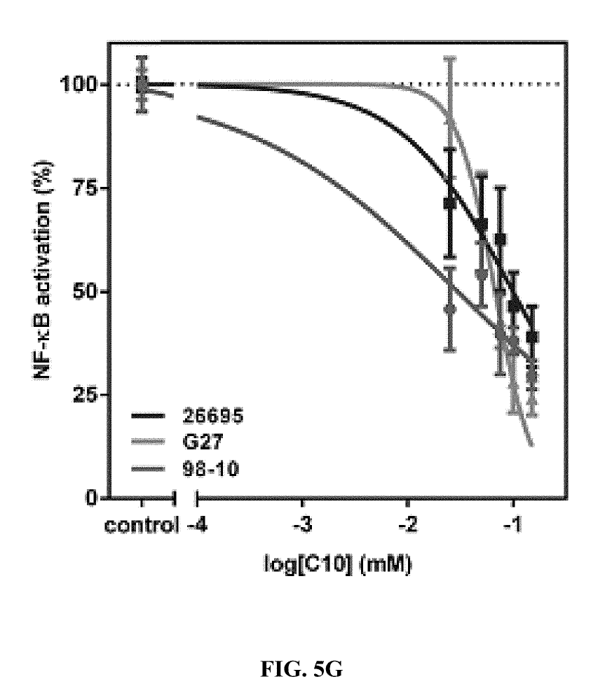

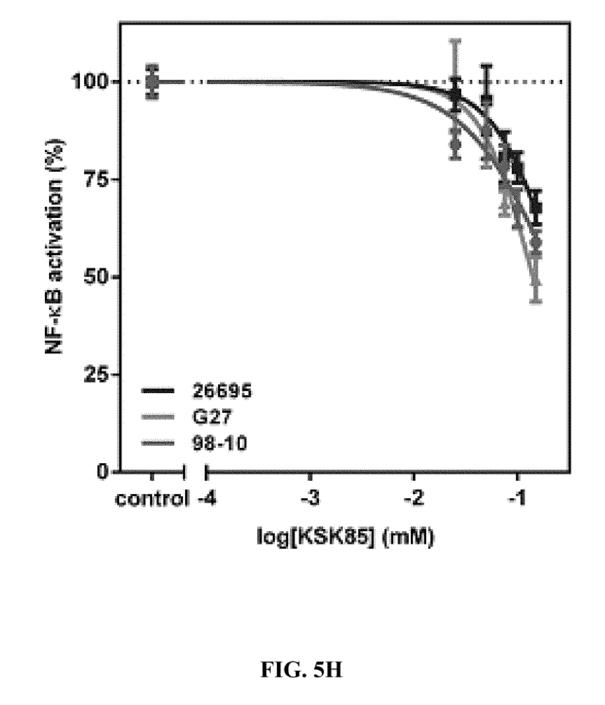

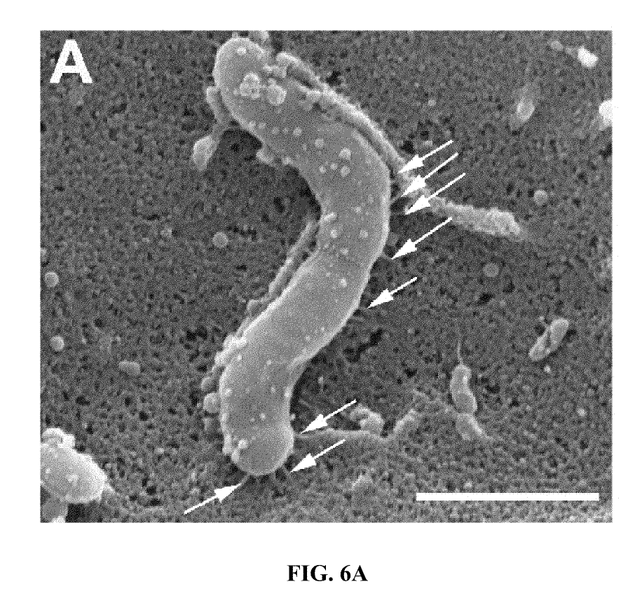

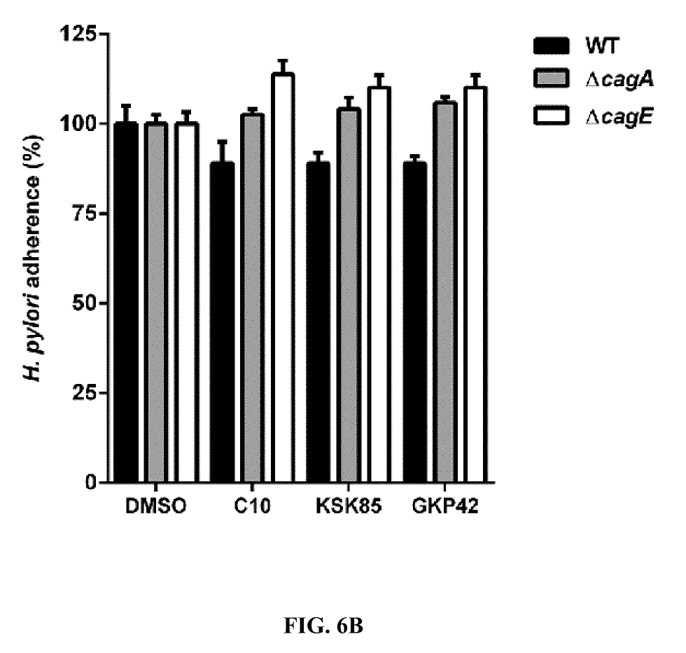

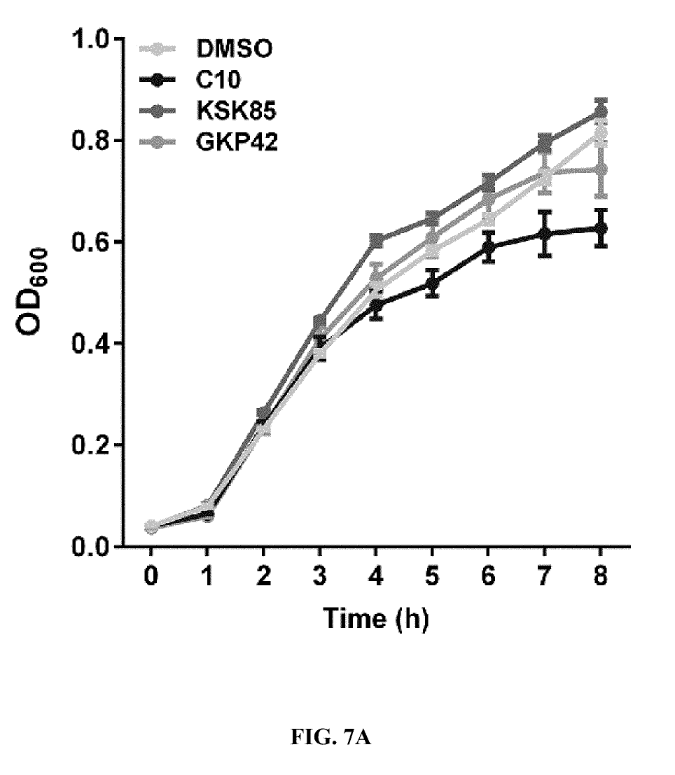

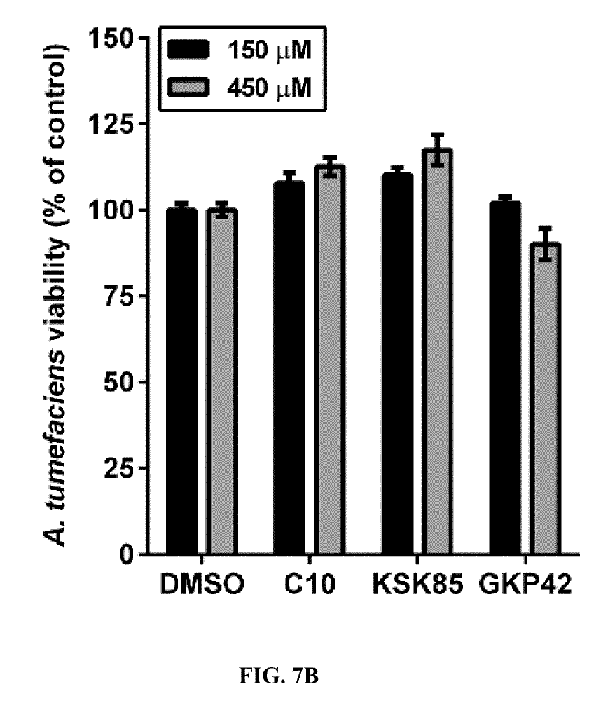

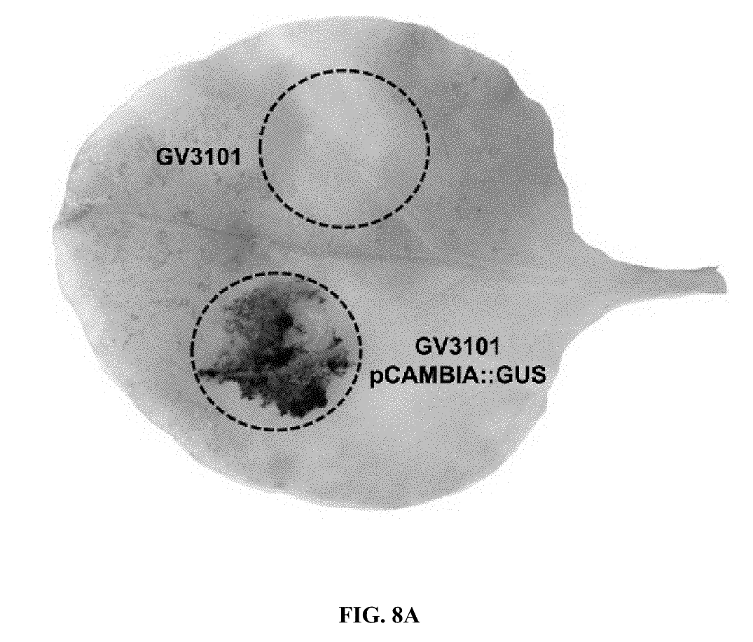

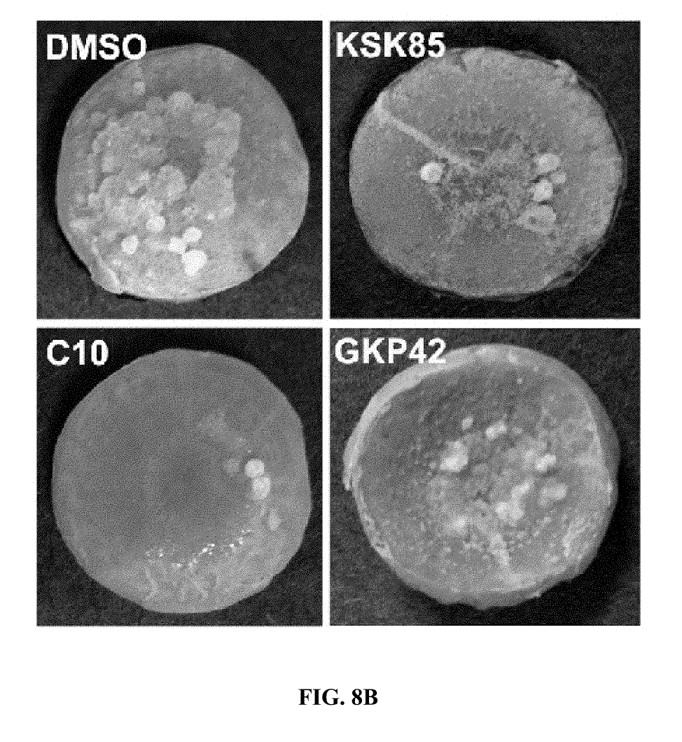

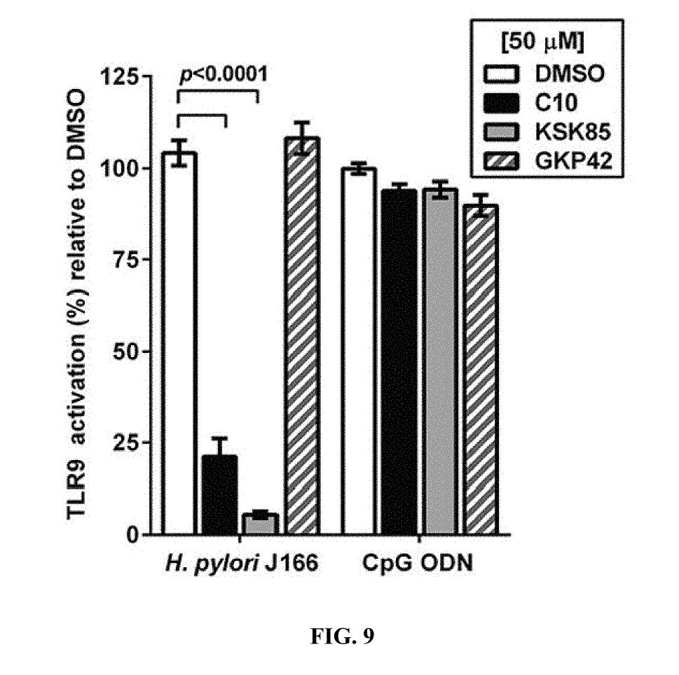

Inhibition of T4SS-dependent activation of human TLR9 signaling by H. pylori. H. pylori strain J166 was grown in broth culture overnight, and was subsequently pre-treated in the indicated compound at a final concentration of 50 μM, or in an equivalent volume of DMSO vehicle for 30 min prior to inoculating cultured HEK293-TLR9 reporter cell lines. Co-cultures were incubated in the presence of 50 μM compound or an equivalent volume of DMSO vehicle supplemented in the cell culture media for the duration of incubation (18 hours). TLR9 activation was assayed using a colorimetric assay (Invivogen), and TLR9 activation in the presence of type IV secretion system (T4SS) inhibitor compounds is expressed as a percentage of TLR9 activation by H. pylori in the presence of vehicle. As a control, TLR9 was activated with the immunostimulatory synthetic ligand CpG oligodeoxynucleotide (CpG ODN) in the presence of DMSO or inhibitor compound at 50 μM final concentration. As shown in FIG. 9, compared to vehicle controls, C10 and KSK85 inhibit T4SS-dependent activation of TLR9 by H. pylori, but not by CpG ODN, indicating that the compounds are able to inhibit or disrupt microbial processes that activate components of the host immune system.

TLR9 activation reporter cell lines (Invivogen) were utilized to screen an SAR library to identify compounds that prevent activation of TLR9 in HEK293 cells via H. pylori cag T4SS activity. H. pylori were grown for 24 hours on blood agar plates, the bacteria were harvested and suspended in sterile phosphate buffered saline (PBS) at an OD600 of 0.5, and were inoculated into wells containing cultured HEK293-TLR9 cell in triplicate at a multiplicity of infection (MOI) of 100 bacterial cells per HEK293-TLR9 cell. Subsequent to bacterial inoculation, cell culture media supplemented with the indicated compound was added to each well at a final concentration of 50 μM. Co-cultures were incubated overnight, and TLR9 activation was assayed approximately 18 hours post-infection as a proxy to measuring H. pylori cag T4SS activity. TLR9 activation in the presence of SAR library compounds was compared to TLR9 activation in wells containing an equivalent volume of DMSO vehicle alone, and is expressed as a percentage of DMSO negative controls.

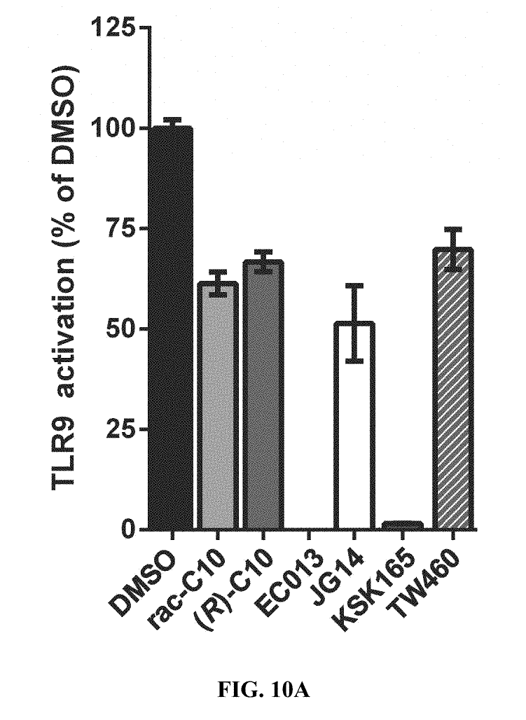

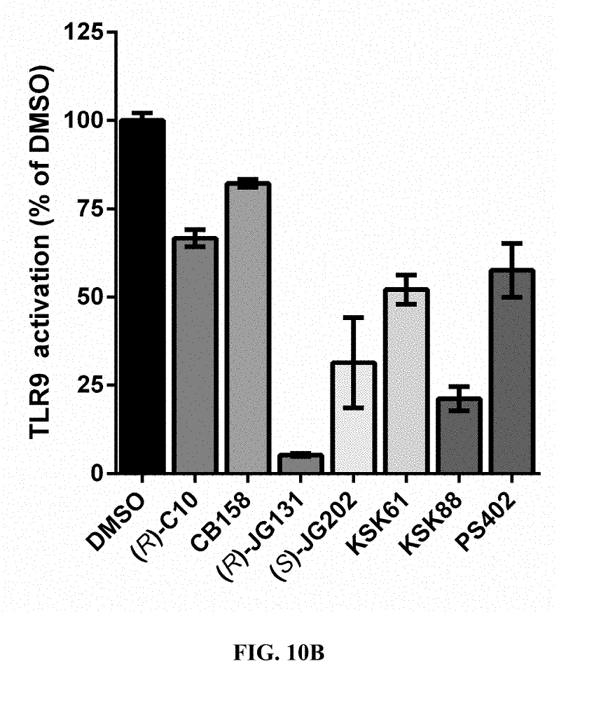

The data is split into 2 graphs (FIGS. 10A and 10B) with compounds grouped according to either C10 or KSK85 derivatives, and both graphs include DMSO vehicle controls (set to 100% H. pylori cag T4SS activity (no inhibition)), and (R)-C10 as a positive control compound. These assays were performed without bacterial cell pre-treatment with the indicated compound. Bacteria were exposed to each compound only during the course of co-culture with HEK293-TLR9 cells, in which the compound was added at the time of bacterial cell inoculation, and was supplemented into the cell culture media (DMEM +10% FBS).

| | | T4SS |
|---|---|---|
| | | Inhibiton |
| | | [50 μM] |
| | | (TLR9 |
| Com- | | Activation |
| pound | Structure | Assay)^{a} |
| rac-C10 | 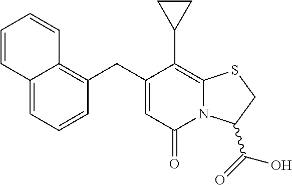 [SMILES: O=C(O)C1CSc2c(C3CC3)c(Cc3cccc4ccccc34)cc(=O)n21] | ++ |
| (R)-C10 | 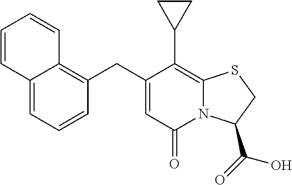 [SMILES: O=C(O)[C@@H]1CSc2c(C3CC3)c(Cc3cccc4ccccc34)cc(=O)n21] | ++ |
| EC013 | 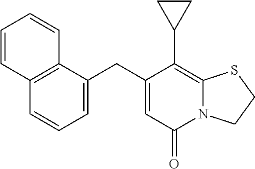 [SMILES: O=c1cc(Cc2cccc3ccccc23)c(C2CC2)c2n1CCS2] | +++ |
| JG14 | 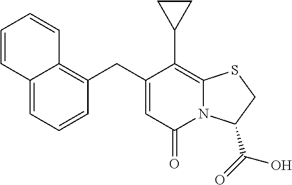 [SMILES: O=C(O)[C@H]1CSc2c(C3CC3)c(Cc3cccc4ccccc34)cc(=O)n21] | ++ |
| KSK165 | 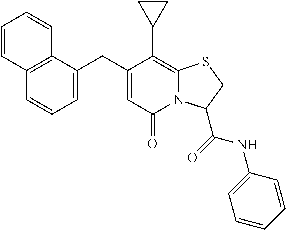 [SMILES: O=C(Nc1ccccc1)C1CSc2c(C3CC3)c(Cc3cccc4ccccc34)cc(=O)n21] | +++ |
| TW460 | 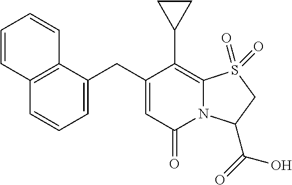 [SMILES: O=C(O)C1CS(=O)(=O)c2c(C3CC3)c(Cc3cccc4ccccc34)cc(=O)n21] | + |
| CB158 | 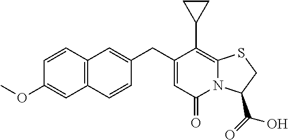 [SMILES: COc1ccc2cc(Cc3cc(=O)n4c(c3C3CC3)SC[C@H]4C(=O)O)ccc2c1] | + |
| (R)- JG131 | 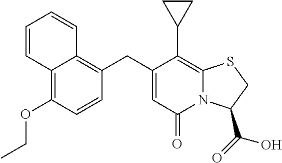 [SMILES: CCOc1ccc(Cc2cc(=O)n3c(c2C2CC2)SC[C@H]3C(=O)O)c2ccccc12] | +++ |
| (S)- JG202 | 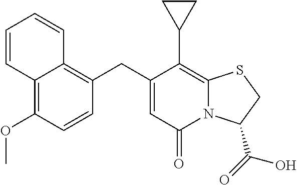 [SMILES: COc1ccc(Cc2cc(=O)n3c(c2C2CC2)SC[C@@H]3C(=O)O)c2ccccc12] | ++ |
| KSK61 | 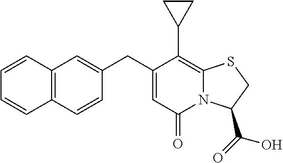 [SMILES: O=C(O)[C@@H]1CSc2c(C3CC3)c(Cc3ccc4ccccc4c3)cc(=O)n21] | ++ |
| KSK88 | 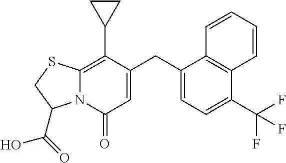 [SMILES: O=C(O)C1CSc2c(C3CC3)c(Cc3ccc(C(F)(F)F)c4ccccc34)cc(=O)n21] | +++ |
| PS402 | 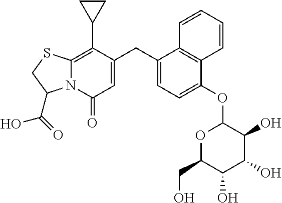 [SMILES: O=C(O)C1CSc2c(C3CC3)c(Cc3ccc(OC4O[C@H](CO)[C@@H](O)[C@@H](O)[C@@H]4O)c4ccccc34)cc(=O)n21] | ++ |

^{a}+++ = Very good T4SS inhibition (reproducibly inhibits T4SS activity by at least 50%, with representative results of about 80% inhibition at higher concentrations); ++ = Moderate T4SS inhibitory effects; + = Low T4SS inhibitory effects. The plus signs qualitatively indicate the inhibitory activity of the test compounds.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A compound of formula (I) 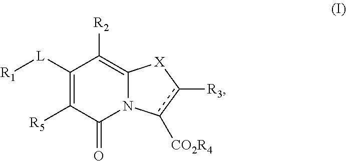
or a salt thereof, wherein
X is —O— —S—, or —S(O)₂—;
R₁ is bicyclic aryl or bicyclic heteroaryl;
L is —O—, —S—, alkylenyl, alkenylenyl, or heteroalkylenyl;
R₂ is cycloalkyl;
R₃ is hydrogen, halogen, or alkyl;
R₄ is hydrogen or alkyl;
R₅ is hydrogen, halogen, alkyl, amino, alkylamino, or dialkylamino; and [symbol]
is a single bond or a double bond;
wherein said bicyclic aryl, bicyclic heteroaryl, alkylenyl, alkenylenyl, heteroalkylenyl, cycloalkyl, and alkyl groups are each optionally substituted with one or more same or different substituents.

2. (canceled)

3. (canceled)

4. The compound of claim 1, or a salt thereof,
wherein:
X is —S—;
L is —CH₂—;
R₁ is a group of formula: 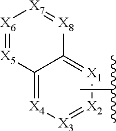
wherein X₁, X₂, X₃, X₄, X₅, X₆, X₇, and X₈ are each independently —CR₆, wherein R₆, at each occurrence, is independently selected from hydrogen, halogen, C₁-C₆-alkyl, C₁-C₆-haloalkyl, C₁-C₆-alkoxy, and C₁-C₆-haloalkoxy;
R₂ is C₃-C₇-cycloalkyl: and
R₃ is hydrogen or optionally substitated C₃-C₆-alkyl.

5. (canceled)

6. The compound of claim 1, or a salt thereof, wherein R₁ is a group of formula: 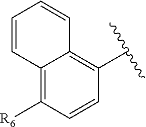
wherein R₆ is hydrogen, halogen, C₁-C₆-alkyl, C₁-C₆-haloalkyl, C₁-C₆-alkoxy, C₁-C₆-haloalkoxy, or

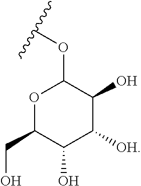 [SMILES: *OC1O[C@H](CO)[C@@H](O)[C@@H](O)[C@@H]1O]

7. The compound of claim 4, or a salt thereof, wherein R₆ is hydrogen or C₁-C₆-alkoxy.

8. (canceled)

9. (canceled)

10. The compound of claim 4, or a salt thereof, wherein R₂ is cyclopropyl, R₄ is hydrogen, and R₅ is hydrogen.

11. (canceled)

12. (canceled)

13. (canceled)

14. The compound of claim 4, or a salt thereof, wherein the compound of formula (I) has formula (I-a),

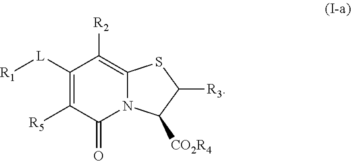

15. The compound of claim 10, or a salt thereof, wherein the compound of formula (I) has formula (I-b),

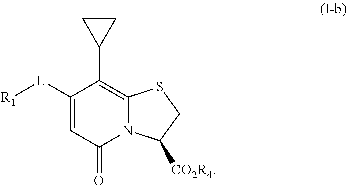

16. The compound of claim 10, or a salt thereof, wherein the compound of formula (I) has formula (I-c),

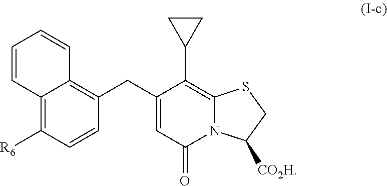

17. The compound of claim 16, or a salt thereof, wherein R₆ is hydrogen.

18. The compound of claim 16, or a salt thereof, wherein R₆ is C₁-C₆-alkoxy.

19. The compound of claim 16, or a salt thereof, wherein R₆ is methoxy.

20. The compound of claim 1, selected from the group consisting of:
(3R)-7-[(4-methoxynaphthalen-1-yl)methyl]-8-cyclopropyl-5-oxo-2,3-dihydro-5H-[1,3]thiazolo[3,2-a]pyridine-3-carboxylic acid (KSK85); and
(3R)-8-cyclopropyl-7-(naphthalen-1-ylmethyl)-5-oxo-3,5-dihydro-2H-thiazolo[3,2-a]pyridine-3-carboxylic acid (C10);
(S)-8-cyclopropyl-7-(naphthalen-1-ylmethyl)-5-oxo-2,3-dihydro-5-H-thiazolo[3,2-a]pyridine-3-carboxylic acid (JG14);
8-cyclopropyl-7-(naphthalen-1-ylmethyl)-5-oxo-2,3-dihydro-5H-thiazolol[3,2-a]pyridine-3-carboxylic acid 1,1-dioxide (TW460);
(R)-8-cyclopropyl-7-((6-methoxynaphthalen-2-yl)methyl)-5-oxo-2,3-dihydro-5H-thiazolo[3,2-a]pyridine-3-carboxylic acid (CB158);
(R)-8-cyclopropyl-7-((4-ethoxynaphthalen-1-yl)methyl)-5-oxo-2,3-dihydro-5H-thiazolo[3,2-a]pyridine-3-carboxylic acid ((R)-JG131);
(S)-8-cyclopropyl-7-((4-methoxynaphthalen-1-yl)methyl)-5-oxo-2,3 -dihydro-5H-thiazolo[3,2-a]pyridine-3-carboxylic acid ((S)-JG202);
(R)-8-cyclopropyl-7-(naphthalen-2-ylmethyl)-5-oxo-2,3-dihydro-5H-thiazolo[3,2-a]pyridine-3-carboxylic acid (KSK61);
8-cyclopropyl-5-oxo-7-((4-(trifluoromethyl)naphthalen-1-yl)methyl)-2,3-dihydro-5H-thiazolo[3,2-a]pyridine-3-carboxylic acid (KSK88); and
8-cyclopropyl-5-oxo-7-((4-(((3S,4R, 5S, 6R)-3,4,5-trihydroxy-6-(hydroxymethyl)tetrahydro-2H-pyran-2-yl)oxy)naphthalen-1-yl)methyl)-2,3-dihydro-5H-thiazolo[3,2-a]pyridine-3-carboxylic acid (PS402);
or a salt thereof.

21. A pharmaceutical composition comprising a therapeutically effective amount of the compound of claim 1, or a pharmaceutically acceptable salt thereof, in combination with one or more pharmaceutically acceptable carriers.

22. An agricultural composition comprising a biologically effective amount of the compound of claim 1, or an acceptable salt thereof, in combination with one or more acceptable carriers.

23. A method for inhibiting the biogenesis and function of bacterial type IV secretion systems, comprising administration of a therapeutically or biologically effective amount of the compound of claim 1, or a salt thereof to a subject in need thereof.

24. A method for inhibiting the transfer of macromolecular substrates (e.g., proteins, DNA) from bacterium to bacterium, or from bacterium to host, comprising administration of a therapeutically or biologically effective amount of the compound of claim 1, or a salt thereof to a subject in need thereof.

25. A method for treating or preventing a condition and disorder caused by or associated with Helicobacter pylori infection, comprising administration of a therapeutically or biologically effective amount of the compound of claim 1, or a salt thereof to a subject in need thereof.

26. (canceled)

27. A method for treating or preventing a condition and disorder caused by or associated with Agrobacterium tumefaciens infection, comprising administration of a therapeutically or biologically effective amount of the compound of claim 1, or a salt thereof to a subject in need thereof.

28. (canceled)

29. A method for preventing or inhibiting transfer of antibiotic resistance among strains of bacteria, comprising administration of a therapeutically or biologically effective amount of the compound of claim 1, or a salt thereof to a subject in need thereof.

30. The method of claim 29, wherein type IV secretion system-mediated gene exchange is prevented or inhibited.
